(19) United States
(12) Patent Application Publication
(10) Pub. No.: US 2024/0279698 A1
(43) Pub. Date: Aug. 22, 2024

(54) SPECIFIC ALPHA-1,2-FUCOSYLTRANSFERASE FOR THE BIOCATALYTIC SYNTHESIS OF 2'-FUCOSYLLACTOSE

(71) Applicant: WACKER CHEMIE AG, Munich (DE)

(72) Inventors: Julia MARTIN, Munich (DE); Carsten BORNHÖVD, Munich (DE)

(73) Assignee: WACKER CHEMIE AG, Munich (DE)

(21) Appl. No.: 18/681,175

(22) PCT Filed: Aug. 5, 2021
(86) PCT No.: PCT/EP2021/071957

§ 371 Date: Feb. 5, 2024

(51) Int. Cl. C12P 19/00; C12N 9/10

(52) U.S. Cl. CPC C12P 19/00; C12N 9/1051; C12Y 204/01069

(57) ABSTRACT

An enzyme that it is a fusion protein. The enzyme includes an N-terminal domain of at least amino acids 1-129 of SEQ ID No. 7 or an amino acid sequence at least 80% identical thereto and that includes at least amino acids 155-286 of SEQ ID No. 5 or an amino acid sequence at least 80% identical thereto as a C-terminal domain and has fucosyltransferase activity. The N-terminal domain and the C-terminal domain being derived from two different fucosyltransferases.

The invention relates to an enzyme, characterized in that it is a fusion protein that comprises (i) an N-terminal domain of a fucosyltransferase and that comprises (ii) at least amino acids 155-286 of SEQ ID No. 5 or an amino acid sequence at least 80% identical thereto as a C-terminal domain and has fucosyltransferase activity, the N-terminal domain and the C-terminal domain being derived from two different fucosyltransferases. The invention further relates to a method for producing 2′-fucosyllactose, characterized in that lactose is in a reaction mixture in the presence of at least one substance selected from the group of substances consisting of glucose, glycerol, sucrose, fucose, and GDP-, ADP-, CDP-, and TDP-fucose reacted with this enzyme.

Thus far, approx. 200 different complex oligosaccharides referred to as human milk oligosaccharides, or HMOs (plural) or HMO (singular) for short, have been identified in human milk. The high diversity arises from the varying combination of the five monosaccharides D-glucose, D-galactose, N-acetyl-D-glucosamine, L-fucose, and N-acetylneuraminic acid into simple and sometimes very complex oligosaccharides. Depending on which monosaccharides an HMO is formed from, a distinction is made between fucosylated neutral, non-fucosylated neutral, and sialylated acidic HMOs (Petschacher and Nidetzky 2016, J. Biotechnol. 235, pp. 61-83).

Unlike the other important constituents of human milk, i.e. sugars such as lactose, lipids, and proteins, HMOs are not metabolized by babies. Instead, they play an important role in the development of a healthy intestinal microbiome, in the prevention of infectious diseases, and in the development of a healthy immune system. HMOs achieve these effects by providing benign bacteria that are able to metabolize HMOs with a growth advantage over pathogens that cannot metabolize HMOs. In addition, they prevent the adhesion of pathogens to the intestinal wall, by mimicking the sugar structures on the epithelial cells to which the pathogens would bind, thereby saturating the surface of the pathogen, which ultimately leads to its excretion. Last but not least, HMOs after they have been absorbed from the intestine also have a direct influence on the gene regulation of intestinal epithelial cells and immune cells, thereby exerting inter alia, via cytokine expression, systemic anti-inflammatory effects (Faijes et al. 2019, Biotechnology Advances 37, pp. 667-697; Petschacher 2018, Hebamme 31, pp. 409-414).

Human milk is distinguished by its high content of HMOs and the complex composition thereof; in some cases certain HMOs occur in significant amounts only in human milk. Thus, although HMOs are detected in other mammals too, they are found only in very low concentrations. Human milk substitutes are accordingly supplemented with HMOs so as to achieve the mentioned beneficial properties. Another emerging field of use is their use as a dietary supplement for adults (Elison et al. 2016, Br. J. Nutr. 116, pp. 1356-1368).

The most common HMO in human milk is the trisaccharide 2′-fucosyllactose, or 2′-FL for short (Deng et al. 2020, Syst. Microbiol. and Biomanuf. 1, pp. 1-14). 2′-FL consists of the monosaccharides D-glucose, D-galactose, and L-fucose, the D-galactose being covalently linked to D-glucose by a β-1,4-glycosidic bond and to L-fucose by a α-1,2-glycosidic bond (Fuc-α1,2-Gal-β1,4-Glc).

Enzymatic processes are an attractive option for the synthesis of HMOs, for example of 2′-FL, since the selective bond formation that is necessary makes a chemical synthesis uneconomical. The regio- and stereoselectivity of enzymes allow synthesis without the use of protecting groups, which is economically advantageous, especially for more complex structures.

In the enzyme-catalyzed synthesis of fucosylated HMOs, fucosyltransferases are generally used. The latter belong to the glycosyltransferase enzyme family (GTs; EC 2.4.) and catalyze the transfer of a fucose unit from a donor, usually guanosine diphosphate fucose, or GDP-fucose for short, to an acceptor, where the latter can be an oligosaccharide, glycoprotein/protein or glycolipid/lipid. The reactive group of the acceptor to which the fucosyltransferase transfers the fucose determines the class of fucosyltransferase. A distinction is made between α-1,2-, α-1,3/4-, and α-1,6-fucosyltransferases. The enzymatic synthesis of 2′-FL employs α-1,2-fucosyltransferases, which transfer the fucose of GDP-fucose to lactose, more precisely the 2′-hydroxyl group of the galactose unit, resulting in the formation of a α-1,2-glycosidic bond. If a nonspecific 1,2-fucosyltransferase is used for this purpose, 2′-FL is formed according to scheme (1) below, it being possible in addition for the 3′-hydroxyl group of the glucose unit also to undergo fucosylation in a nonspecific manner, resulting in the formation of the by-product 2,3-difucosyllactose, more precisely Fuc-α1,2-Gal-β1,4-(Fuc-α1,3-)Glc:
(1) Lactose+GDP-fucose→2′-FL+difucosyllactose

With a specific 1,2-fucosyltransferase, 2′-FL is by contrast formed according to scheme (2), without formation of the undesirable by-product:
(2) Lactose+GDP-fucose→2′-FL

Although fucosyltransferases belong to the glycosyltransferase class (EC 2.4.), they are just one example of enzymes of this class. In general, glycosyltransferases catalyze the transfer of a sugar molecule from a donor to an acceptor. Although the sequence homology between different GTs is low, the majority of GTs can be assigned to one of two structural superfamilies, namely GT-A and GT-B. What the two superfamilies have in common that the enzymes consist of two domains connected to one other by a linker structure/sequence. The active center of the enzyme is here formed from regions of the two domains and is located therebetween.

Enzymes of the GT-A family have an N-terminal domain consisting of β-pleated sheets that are in each case surrounded by α-helices (in what is known as a Rossmann fold), it being this domain that recognizes the donor, whereas the C-terminal domain consists mainly of mixed beta-pleated sheets and binds the acceptor.

In contrast, enzymes of the GT-B family have two Rossmann fold-type folded structures. Whereas the N-terminal domain forms the acceptor binding site, the C-terminal structure is responsible for the binding of the donor. Presumably due to the lower variability of donor sugars compared to the wide range of acceptor sugars, the C-terminal domains of different glycosyltransferases of the GT-B family are more highly conserved than the N-terminal domains (Albesa-Jové et al. 2014, Glycobiology 24, pp. 108-124).

Because of the conserved folding within a structural superfamily, such as the GT-B family, it is possible to combine the domains of two different glycosyltransferases of a structural superfamily with one another. The exchange of similarly folded protein domains of different origins, also known as domain swapping, is a commonly used method in enzyme characterization and in metabolic engineering and allows the generation of hybrid enzymes having novel properties (for example altered activities or substrate specificities, etc.) (Schmid et al. 2016, Front. Microbiol. 7, 182, pp. 1-7; Hansen et al. 2009, Phytochemistry 70, pp. 473-482; Park et al. 2009, Biotechnol. Bioeng. 102, pp. 988-994; Truman et al. 2009, Chem. Biol. 16, pp. 676-685). However, the results of previous domain swapping experiments in glycosyltransferases are inconsistent. On the one hand, the acceptor or donor specificity of a glycosyltransferase were altered by the exchange of the corresponding domains (Truman et al. 2009, Chem. Biol. 16, pp. 676-685); on the other hand, both the C- and the N-terminal domain influenced the specificity for the acceptor such that prediction of substrate specificities is not usually possible (Hansen et al. 2009, Phytochemistry 70, pp. 473-482). This is surely also because the active center in this enzyme class is formed by the positioning of the two domains next to one another, with the result that minor differences in the three-dimensional structure often lead to inactive enzymes or at least to distorted binding sites. Such experiments can therefore as a rule be expected to afford enzymes that are inactive or that do not have the desired specificity and reactivity.

Besides specificity and activity, protein stability and solubility play a major role in fucosyltransferases. For example, in the expression of fucosyltransferases in E. coli, the formation of inclusion bodies (Lee et al. 2015, Microbiology and Biotechnology Letters 43, pp. 212-218) and low protein stability (Wang et al. 1999, Microbiology (Reading) 145, pp. 3245-3253) have frequently been observed. Repeated attempts have therefore been made to increase the solubility/stability and folding of fucosyltransferases. As well as coexpression of chaperones (Lee et al. 2015, Microbiology and Biotechnology Letters 43, pp. 212-218), there is the option of translational fusion of the fucosyltransferase with a quick-folding and highly soluble protein such as glutathione-S-transferase (GST) (Albermann et al. 2001, Carbohydr. Res. 334, pp. 97-103). Alternatively, the solubility can be increased by appending charged amino acids such as a negatively charged aspartate tag (Chin et al. 2015, J. Biotechnol. 210, pp. 107-115). There are also approaches aimed at improving protein stability by using an amino acid consensus sequence formed from a plurality of homologous proteins (Porebski and Buckle 2016, Protein Eng. Des. Sel. 29, pp. 245-251). This approach takes account of the evolutionary information represented by the homologous sequences and is based on the hypothesis that a conserved amino acid contributes more to stability than one that is unconserved (Steipe et al. 1994, J. Mol. Biol. 240, pp. 188-192).

The synthesis of 2′-FL using various α-1,2-fucosyltransferases up to the point of industrial implementation had already been demonstrated prior to the invention described herein. These include inter alia the α-1,2-fucosyltransferase from Helicobacter pylori UA802 (futC, GenBank AF076779; EP1243674, Kyowa Hakko, α-1,2-fucosyltransferase from 1990), the Helicobacter mustelae NCTC12198/ATCC43772 (futL, GenBank CBG40460.1; EP1426441, Kyowa Hakko, 2001), the α-1,2-fucosyltransferase from E. coli of serogroup O126 (wbgL, Engels and Elling 2014, Glycobiology 24, pp. 170-178), and the α-1,2-fucosyltransferase from Bacteroides fragilis (wcfB, Chin et al. 2017, J. Biotechnol. 257, pp. 192-198). In a comparison of the above enzymes in respect of 2′-FL yield in batch fermentation of E. coli strains for production of 2′-FL, the highest yield was with futC from H. pylori UA802, which was therefore a pointer to the high activity of this enzyme (Huang et al. 2017, Metab. Eng. 41, pp. 23-38).

Some sequence modifications were also made for more exact characterization and optimization of the enzymes. Analyses of futC from H. pylori UA802 showed that shortening the N-terminus by using two alternative start codons (Δaa 1-15, i.e. starting with amino acid M16; or Δaa 1-46, i.e. starting with amino acid M47) resulted in complete loss of activity (Wang et al. 1999, Microbiology (Reading) 145, pp. 3245-3253). Likewise, the insertion of a resistance gene upstream of conserved region aa 163-173, more precisely downstream of aa 152 of the futC gene, resulted in a loss of function in respect of production of the fucosylated Lewis Y antigen in H. pylori UA802 (Wang et al. 1999, Mol. Microbiol. 31, 1265-1274). These experiments showed that the entire coding sequence is necessary for fucosyltransferase activity and that even small manipulations of the amino acid sequence can result in complete inactivation of the enzyme.

Because futC from H. pylori UA802 accepts not just lactose but also monofucosylated sugars as substrates (Wang et al. 1999, Microbiology (Reading) 145, pp. 3245-3253), the synthesis of 2′-FL is accompanied, as a consequence of additional fucosylation of the 3′-hydroxyl group of the glucose unit, by formation of the by-product difucosyllactose (DFL) or lactodifucotetraose (LDFT), more precisely Fuc-α1,2-Gal-β1,4-(Fuc-α1,3-) Glc, (Yu et al. 2018, Microb. Cell. Fact. 17, 101, pp. 1-10). This was also demonstrated for the α-1,2-fucosyltransferase from H. pylori strain 26695 (Chin et al. 2017, J. Biotechnol. 257, pp. 192-198).

DFL formation in such a production batch has a doubly negative effect on the efficiency of the synthesis of 2′-FL, since not only is 2′-FL lost as a result of conversion into DFL but the activated fucose (GDP-fucose) that is needed is consumed too. The latter is then no longer available for the synthesis of 2′-FL. In addition, the DFL formed makes it difficult to work up the fermentation culture into pure 2′-FL, because of the very similar physical and chemical properties of 2′-FL and DFL.

In a preferred embodiment, preference is therefore given to using a regio- and substrate-specific α-1,2-fucosyltransferase for the specific fucosylation of the 2′-hydroxyl group of the galactose unit of lactose. An example that has been identified is the α-1,2-fucosyltransferase futL from H. mustelae NCTC12198/ATCC43772, which, alongside significantly reduced synthesis of the by-product DFL, forms 2′-FL specifically (EP2877574, Glycosyn, 2015). As a result of the described high activity and specificity toward lactose compared to futC from H. pylori UA802, it has likewise already been used for the synthesis of 2′-FL (EP1426441, Kyowa Hakko, 2001). On the other hand, a direct comparison of futL with a futC similar to that of H. pylori UA1210 in respect of 2′-FL yield under the same batch fermentation conditions (Huang et al. 2017, Metab. Eng. 41, pp. 23-38) showed that futL achieved only about 75% of the yield that was attained with futC.

In the CAZY database (www.cazy.org/GT11_bacteria.html), both 1,2-fucosyltransferases, i.e. futC from H. pylori UA802 and futL from H. mustelae NCTC12198/ATCC43772, are assigned to glycosyltransferase family 11 (GT-11) (Ma et al. 2006, Glycobiology 16, pp. 158-184). Although it has previously been predicted for the GT-11 family, by Breton et al. 2012, Curr. Opin. Struct. Biol. 22, pp. 540-549, that the enzymes classified therein could exhibit GT-B folding and this should continue to be the assumption (Petschacher and Nidetzky 2016, J. Biotechnol. 235, pp. 61-83), it has to date not been possible to unambiguously assign members of the GT11 family to either the GT-B or GT-A fold family (Schmid et al. 2016, Front. Microbiol. 7, 182, pp. 1-7), which makes correct prediction of donor/acceptor specificities for domain swapping experiments additionally improbable.

According to the presented prior art, it would be expected to be the N-terminal domain of the specific (for the definition of specific see scheme 2) futL from H. mustelae NCTC12198/ATCC43772 that—given the presumed assignment to the GT-B superfamily—is responsible for the binding of the acceptor and thus responsible for the fucosylation of lactose to 2′-FL without formation of the by-product DFL.

The object of this invention was to increase the efficiency of the biocatalytic synthesis of 2′-FL, i.e. to achieve an increased yield of 2′-FL and at the same time avoid the formation of the very similar by-product DFL, so as to facilitate workup of the 2′-FL and thus make it possible to establish an economic industrial process.

The object was achieved by providing an enzyme, characterized in that it is a fusion protein that comprises (i) an N-terminal domain of a fucosyltransferase and that comprises (ii) at least amino acids 155-286 of SEQ ID No. 5 or an amino acid sequence at least 80%, preferably at least 90%, and more preferably at least 95%, identical thereto as a C-terminal domain and has fucosyltransferase activity, the N-terminal domain and the C-terminal domain being derived from two different fucosyltransferases. This fusion protein has surprisingly succeeded in combining the substrate- and regiospecificity of the α-1,2-fucosyltransferase futL with the potentially higher activity of the enzyme futC. This could not have been predicted from the current prior art, since it would have been expected therefrom to be the N-terminal domain of SEQ ID No. 5 and not the C-terminal domain of SEQ ID No. 5 that is responsible for the specific binding to the fusion protein of the substrate lactose that prevents 2′-FL from being fucosylated further to DFL. An enzyme is thereby provided that can be employed in an economically effective method for the specific fucosylation of lactose to 2′-fucosyllactose.

The fusion protein of the invention constitutes a fusion of the amino acid sequences of the N-terminal domain (protein i) and C-terminal domain (protein ii), where the N-terminal domain and the C-terminal domain originate from two different fucosyltransferases. The wild-type proteins futL (futL from Helicobacter mustelae NCTC12198, SEQ ID No. 5) or futC (for example futC from H. pylori UA802, SEQ ID No. 3) used as examples are not encompassed by the claim, since they are not fusion proteins from two different fucosyltransferases.

The term fusion protein means that the corresponding amino acid sequences and/or coding DNA sequences have been fused in the laboratory and thus do not occur in nature. The fusion protein is also referred to as a hybrid or hybrid enzyme. It can for example be composed of the N-terminal domain of a α-1,2-fucosyltransferase derived from a consensus sequence futC* based on the futC sequence of Helicobacter pylori UA802 and the C-terminal domain of the fut sequence of Helicobacter mustelae NCTC12198. The hybrid enzyme has high activity and specificity and efficiently transfers exclusively fucose to lactose, e.g. fucose to the 2′-hydroxyl group of the galactose unit of the acceptor lactose, but not to the 3′-hydroxyl group of the glucose unit of lactose. It is crucial that the formation of the undesired by-product difucosyllactose is thereby significantly reduced. This makes isolation of the 2′-fucosyllactose much easier and economically more efficient, since laborious purification steps to remove DFL can be largely or completely dispensed with.

A further advantage of using a fusion protein is that, through the choice of the N- and C-terminal domain, it is possible to vary the acceptance for the donor and acceptor and in doing so also make it possible to produce more complex HMOs more efficiently. In addition, a higher enzyme activity, such as that of futC, can be combined with a better selectivity for the acceptor substrate, such as that of futL.

In order to combine the presumably higher activity of futC with the better selectivity of futL for the acceptor substrate, the N-terminal domain of futC* (aa 1-148 of SEQ ID No. 7) was fused with the C-terminal domain of futL (aa 142-286 of SEQ ID No. 5) or, in the reverse combination, the N-terminal domain of futL (aa 1-143 of SEQ ID No. 5) was fused with the C-terminal domain of futC* (aa 151-300 of SEQ ID No. 7). For this, an Scal-(AGTACT) cut site was introduced into the linker sequence between the two enzyme domains by exchanging bases 426-427 (TA to CT) in the nucleic acid sequence of the futL gene (SEQ ID No. 4) on the plasmid encoded for futL, the amino acid sequence remaining unchanged by this exchange. Through the introduction of this cut site it was possible, with the aid of this plasmid construct, by restriction digestion with suitable restriction enzymes that cleave terminally (EcoRI/XbaI) or between domains (Scal), to execute an exchange of the N-terminal/C-terminal domain of futL with an appropriate PCR-amplified domain of another, for example of futC* (see example 2). The resulting cds for the futC*/futL (SEQ ID No. 8) or futL/futC* (SEQ ID No. 10) hybrid on the low-copy expression plasmid were transcriptionally combined in an operon with optimized RBS with the cds for resA (SEQ ID No. 18).

The enzyme of the invention has fucosyltransferase (FT) activity, i.e. it catalyzes the transfer of a fucose unit from a donor such as GDP-, ADP-, CDP- or TDP-fucose, preferably guanosine diphosphate fucose (GDP-fucose), which can be formed starting from glycerol, sucrose, glucose or fucose, to an acceptor, the latter being an oligosaccharide, such as preferably lactose, or a glycoprotein, protein, glycolipid or lipid.

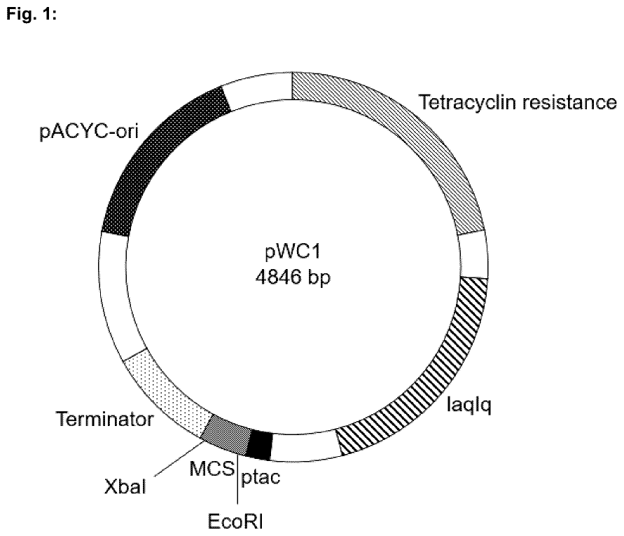

For the detection of fucosyltransferase activity, the cds of the test protein with a codon usage optimized for E. coli is amplified by PCR with appropriate oligonucleotides and cloned by means of appended cut sites into an expression plasmid such as the low-copy expression plasmid pWC1 downstream of a promoter, preferably an inducible promoter (see e.g. example 2; FIG. 1).

Suitable promoters are all promoters known to those skilled in the art, such as constitutive promoters, for example the GAPDH promoter, or inducible promoters, for example the lac, tac, trc, T7, lambda PL, ara, cumate or tet promoter or sequences derived therefrom. Preferably, the promoter controlling the expression of the enzyme of the invention is an inducible promoter, more preferably a promoter induced by IPTG (isopropyl-ß-D-thiogalactopyranoside).

When the host cell is an E. coli cell, it is preferable that the cds for the E. coli endogenous transcription activator rcsA (SEQ ID No. 18 (DNA)/SEQ ID No. 19 (PRT)) of the de-novo pathway for GDP-fucose with optimized ribosome binding site (RBS) is polycistronically inserted into the finished expression plasmid downstream of the respective cds of the fucosyltransferase, in order to increase the intracellular endogenous production of GDP-fucose on the de-novo pathway.

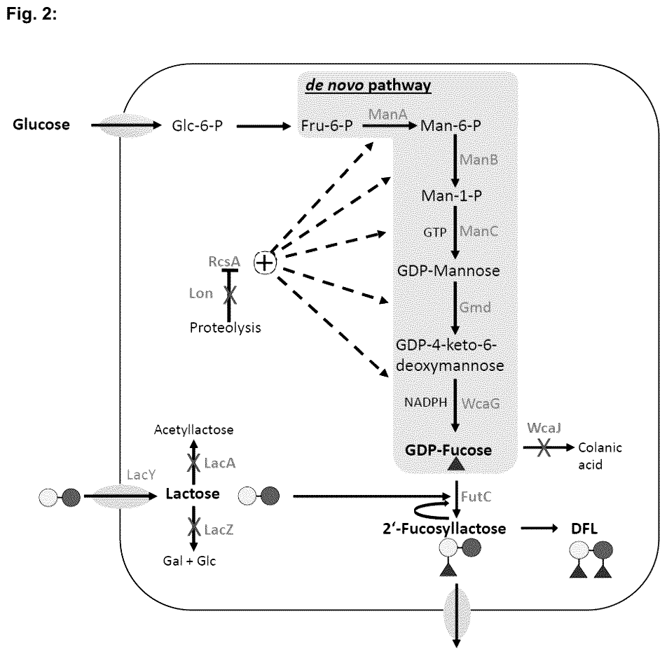

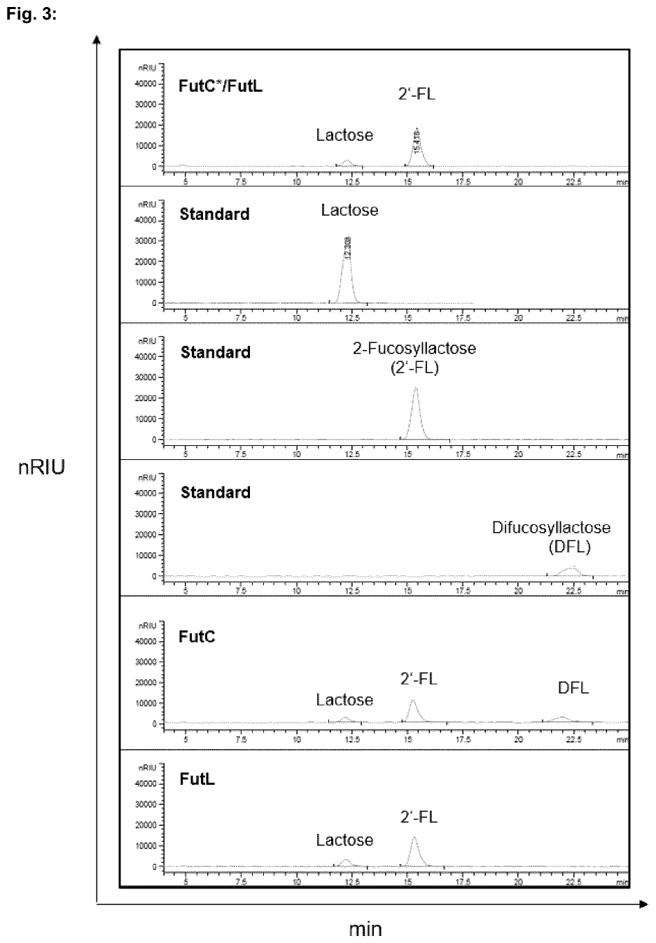

Through transformation of a microbial strain that is able to provide GDP-fucose or another activated fucose (such as ADP-, GDP-, CDP- or TDP-fucose) as donor intracellularly, such as preferably an appropriate E. coli strain such as E. coli K12ΔwcaJΔIonΔsulA-lac-mod (see example 1 and FIG. 2) with the expression plasmid, the person skilled in the art will obtain a strain that differs only in the expressed fucosyltransferase futC (SEQ ID No. 3), futC* (SEQ ID No. 7), futL (SEQ ID No 5), futC*/futL hybrid (SEQ ID No 9) or futL/futC* hybrid (SEQ ID No 11)) and that can therefore be used for the analysis of FT activity. For this, the resulting strain, which is able to provide the donor intracellularly, is cultured in the presence of a donor precursor such as glycerol, sucrose, glucose or fucose, the strain being able to convert this into GDP-fucose intracellularly, and an acceptor such as lactose in the culture medium. The cds is expressed constitutively or after induction, if an inducible promoter had been selected. As demonstration of the FT activity of the strain, the concentration of the fucosylation product 2′-FL is determined by HPLC. For this, a 1 ml aliquot is taken from the corresponding cell culture having a cell density OD₆₀₀ of at least approx. 160, then all solid constituents are removed, for example by centrifuging for 5 minutes at maximum speed in a bench centrifuge, and the product content of the supernatant obtained is quantified, for example by HPLC as described in example 4 (see also FIG. 3).

The coding sequence (cds) for the different fucosyltransferases used as the starting sequences, are known in the prior art and from databases and can optionally be produced synthetically with a codon usage optimized for the host organism such as E. coli or amplified from the genome of the original organisms by PCR with appropriate oligonucleotides.

What is referred to as the coding sequence (cds) is the region of the DNA or RNA that lies between a start codon and a stop codon and codes for the amino acid sequence of a protein.

Cds are surrounded by non-coding regions. What is referred to as a gene is the section of DNA that contains all the information for producing a biologically active RNA. Thus, a gene contains not only the section of DNA from which a single-stranded RNA copy is produced by transcription, but also additional sections of DNA involved in the regulation of this copying process.

The preferred expression signals that regulate the expression of the cds for the enzyme of the invention include at least one promoter, a transcription start, a translation start, a ribosome binding site, and a terminator. These are particularly preferably functional in the employed bacterial strain, especially preferably in E. coli. For a functional promoter, it is therefore the case that the coding sequences under the regulation of this promoter are transcribed into an RNA.

What is referred to as a wild-type (wt) cds is the form of a cds that occurs naturally through evolution and is present in the wild-type genome of the organism occurring in nature.

A domain or folding class refers to a region having a stably folded, usually compact tertiary structure within a protein. As described in detail in the prior art and elucidated above, all GTs that have a GT-A or GT-B fold consist of an N-terminal domain and a C-terminal domain connected to each other by a linker structure/sequence, the active center being formed from regions of both domains. The 3Dee database (dundee.ac.uk) can by way of example be used to define protein domains.

The names futL and futC refer to the corresponding wild-type fucosyltransferases respectively having SEQ ID No. 5 and/or SEQ ID No. 3, each consisting of an N-terminal and C-terminal domain.

The term futC* refers to a sequence derived from futC having SEQ ID No. 7. It likewise consists of two domains, an N-terminal and a C-terminal domain. futL, futC, and futC* are not fusion proteins.

On the other hand, the name N/C refers to an FT comprising an N-terminal domain of one FT and a C-terminal domain of another FT. Thus, for example, futC*/futL comprises the N-terminal domain of the fucosyltransferase futC* (at least amino acids 1-129 from SEQ ID No. 7 or an at least 80% identical amino acid sequence) and the C-terminal domain of the fucosyltransferase futL (at least amino acids 155-286 from SEQ ID No. 5 or an at least 80% identical amino acid sequence). Similarly, in futL/futC* the N-terminal domain of futL and C-terminal domain of futC* have been fused.

futC*/futL(Δ8aa) and futC*/futL(Δ15aa) comprise the N-terminal domain of futC* and the C-terminal domain of futL, where the C-terminal domain has been shortened by 8 and 15 amino acids respectively.

A homologous amino acid sequence is to be understood as meaning a sequence that is at least 80%, preferably at least 90%, and more preferably at least 95%, identical, each alteration in the homologous sequence being selected from insertion, addition, deletion, and substitution of one or more amino acids.

The identity of the amino acid sequences is determined by the program “Protein blast” on the publicly accessible webpage http://blast.ncbi.nlm.nih.gov/. This program uses the blastp algorithm. The following general parameters are used as algorithm parameters for an alignment of two or more protein sequences: Max target sequences=100; Short queries=“Automatically adjust parameters for short input sequences”; Expect Threshold=10; Word size=3; Max matches in a query range=0. The default scoring parameters are: Matrix=BLOSUM62; Gap Costs=Existence: 11 Extension: 1; Compositional adjustments=“Conditional compositional score matrix adjustment”. For the identification of homologous sequences, the above parameters were used for the search in the “Non-redundant protein sequences (nr)” database, sequences from the organism Helicobacter pylori (taxid: 210) having been excluded on account of high data density at a homology of >80%.

A distinction is made between α-1,2-, α-1,3/4-, and α-1,6-fucosyltransferases. Preferably, the fusion protein of the invention is an enzyme having α-1,2-fucosyltransferase activity.

Preferably, the enzyme is characterized in that the amino acid sequences of the N-terminal and C-terminal domains of the fusion protein are microbial sequences, more preferably sequences of gram-negative bacteria, and especially preferably sequences of a bacterial strain of the genus Helicobacter or a sequence homologous thereto.

In a preferred embodiment, the enzyme is characterized in that the amino acid sequences of the N-terminal and C-terminal domains of the fusion protein are sequences of glycosyltransferase family 11 (GT-11).

Further preferably, the enzyme is characterized in that the amino acid sequences of the N-terminal and C-terminal domain of the fusion protein are sequences of the species Helicobacter pylori or Helicobacter mustelae or a sequence homologous thereto. Particularly preferably, the fusion protein comprises a C-terminal domain from futL of the organism Helicobacter mustelae NCTC12198/ATCC43772 (SEQ ID No. 5). The other, N-terminal, domain of the fusion protein is preferably derived from futC of the organism Helicobacter pylori UA802 (SEQ ID No. 3).

In a preferred embodiment, the enzyme is characterized in that the N-terminal domain comprises at least amino acids 1-129, more preferably at least amino acids 1-132, and especially preferably at least amino acids 1-148, of SEQ ID No. 7, or an amino acid sequence in each case at least 80% identical thereto. In a particularly preferred embodiment, the enzyme is characterized in that the amino acid sequence of the N-terminal domain of the fusion protein is amino acids 1-129 of SEQ ID No. 7, further preferably amino acids 1-132 thereof, and even further preferably amino acids 1-148 thereof, or an amino acid sequence at least 80% identical thereto.

In a preferred embodiment, the enzyme is characterized in that the C-terminal domain comprises at least amino acids 155-286, more preferably at least amino acids 149-286, and especially preferably at least amino acids 142-286, of SEQ ID No. 5, or an amino acid sequence at least 80% identical thereto. Especially preferably, the enzyme is characterized in that the amino acid sequence of the C-terminal domain of the fusion protein is amino acids 155-286 of SEQ ID No. 5, further preferably amino acids 149-286 thereof, and even further preferably amino acids 142-286 thereof, or an amino acid sequence in each case at least 80% identical thereto.

It is preferable that the fusion protein is SEQ ID No. 9, SEQ ID No. 13, SEQ ID No. 15 or an amino acid sequence at least 80% identical thereto. Particularly preferably, the fusion protein is futC*/futL having SEQ ID No. 9.

The 2′-FL yields after fermentation for 65 h at 25° C. from induction and a total lactose input of 65 g/l (batch and continuous) showed that both futC and futC* formed 2′-FL and DFL, the 2′-FL yield for futC being 70% higher than that for futC* (see example 3, Table 1). As described in the literature, futL formed exclusively 2′-FL. The 2′-FL yield for futL exceeded that for futC* by 125% and that for futC by 32%.

The analysis of the yields of 2′-FL and DFL in the expression of the fusion proteins surprisingly showed, contrary to the prior art, that the fusion protein futC*/futL, but not the variant futL/futC*, converted lactose and GDP-fucose 100% specifically into 2′-FL. Furthermore, Table 1 shows that the specific fusion protein futC*/futL increased the 2′-FL yield by 56% compared to the nonspecific futC, by 165% compared to futC*, and by 18% compared to the specific futL. This could not have been predicted, since firstly there is so far no 3-D structure available for either futC or futL and, secondly, assuming a GT-B fold in which the N-terminal domain is responsible for binding the acceptor substrate, it would have been expected to be the fusion protein futL/futC*, which contained the N-terminal domain of the lactose-specific futL, and not futC*/futL, that converts exclusively lactose and GDP-fucose specifically into 2′-FL. Moreover, the 2′-FL yield for futL/futC* was reduced by 70% compared to futC*/futL.

Since the yield with the futC* (SEQ ID No. 7) used for the fusion protein had been reduced by 41% compared to futC (SEQ ID No. 3), the N-terminal domain of futC (aa 1-148 of SEQ ID No. 3) was, as described previously, ultimately fused with the C-terminal domain of futL (aa 142-286 of SEQ ID No. 5) to afford futC/futL (SEQ ID No. 12 (DNA)/SEQ ID No. 13 (PRT)) and the resulting expression plasmid used for the transformation of the strain suitable for 2′-FL production (E. coli K12ΔwcaJΔIonΔsulA-lac-mod) (see examples 1 and 2). The 2′-FL and DFL yields for futC*/futL and futC/futL under optimized fermentation conditions (27° C. instead of 25° C., 86 g/l lactose instead of 65 g/l lactose) showed that no DFL was formed in this case too. The 2′-FL yield with the fusion protein futC/futL was however reduced by 15% compared to the fusion protein futC*/futL (Table 1).

Nevertheless, in both cases the fusion of the N-terminal domain of futC* and of futC with the C-terminal domain of futL resulted in selective production of 2′-FL without formation of the very similar by-product DFL.

In addition, the C-terminus of the hybrid enzyme futC*/futL was shortened by 8 aa (aa 1-285 of SEQ ID No. 9) and 15 aa (aa 1-278 of SEQ ID No. 9) respectively (see example 2) and this was likewise investigated in respect of 2′-FL yield after fermentation for 65 h under optimized conditions (27° C. from induction, 86 g/l lactose). Whereas shortening by 8 aa reduced the 2′-FL yield by 8%, shortening by 15 aa resulted in neither 2′-FL nor DFL being detectable (Table 1). This showed that at least aa 1-285 of the fusion protein futC*/futL are responsible for the activity thereof.

The present invention further provides a method for producing 2′-fucosyllactose, characterized in that lactose is in a reaction mixture in the presence of at least one substance selected from the group of substances consisting of glucose, glycerol, sucrose, fucose, and GDP-, ADP-, CDP-, and TDP-fucose reacted with the enzyme of the invention. The substance undergoing intracellular conversion into GDP-fucose is preferably glucose. The enzyme of invention is preferably futC*/futL having SEQ ID No. 9. Particularly preferably, the enzyme is futC*/futL and the substance undergoing intracellular conversion into GDP-fucose is glucose. In the method of the invention, lactose is able to undergo complete conversion without difucosyllactose being formed.

Preferably, the method for producing 2′-fucosyllactose is characterized in that 2′-fucosyllactose is isolated from the reaction mixture. For the isolation of 2′-fucosyllactose, it is preferable when solid constituents are in a first step removed from the reaction mixture by centrifugation or filtration. In a subsequent step, further impurities can for example be subsequently separated by chromatographic methods and by filtration and 2′-fucosyllactose obtained by evaporation.

In a preferred embodiment, the method is characterized in that lactose undergoes complete conversion without more than 5%, more preferably 2.5%, and especially preferably 1.5%, of DFL being formed; in an especially preferred embodiment, lactose undergoes complete conversion without formation of DFL. The method of the invention thus has the major advantage that the specific formation of 2′-FL eliminates the need to remove other sugars such as DFL or lactose by crystallization or nanofiltration or enzymatic workup. The selective production of 2′-FL accordingly makes workup significantly easier.

In a particularly preferred embodiment, the method is therefore characterized in that 2′-fucosyllactose is isolated without a crystallization, nanofiltration and/or enzymatic workup to remove other sugars such as glucose, lactose or difucosyllactose.

In a preferred embodiment, the method for producing 2′-fucosyllactose is characterized in that the reaction mixture is a culture of microorganisms that recombinantly express the enzyme of the invention.

The culturing of microorganisms is known in the prior art and can by way of example be carried out as described in example 3.

The microorganism strain is particularly preferably a genetically modified E. coli K12 strain. It is likewise preferable that the recombinantly expressed enzyme of the invention is the fusion protein futC*/futL or an amino acid sequence homologous thereto. In an especially preferred embodiment, the microorganism strain is therefore a genetically modified E. coli K12 strain and the recombinantly expressed enzyme of the invention is the fusion protein futC*/futL, more preferably in co-expression of rcsA.

When the reaction mixture is a culture of microorganisms, it is preferable that 2′-fucosyllactose is isolated from the culture supernatant. As already described above, the solid constituents such as the host cells are first separated by filtration or more preferably by centrifugation. Further impurities can subsequently be removed chromatographically and the product obtained in crystalline form by concentration.

As already described above, the method is preferably characterized in that lactose undergoes complete conversion without more than 5%, more preferably 2.5%, and especially preferably 1.5%, of DFL being formed. In an especially preferred embodiment, lactose undergoes complete conversion without formation of DFL. Particularly preferably, 2′-fucosyllactose is isolated from the culture supernatant without a crystallization, nanofiltration and/or enzymatic workup of the fermentation broth to remove other sugars such as glucose, lactose, and difucosyllactose from the culture supernatant.

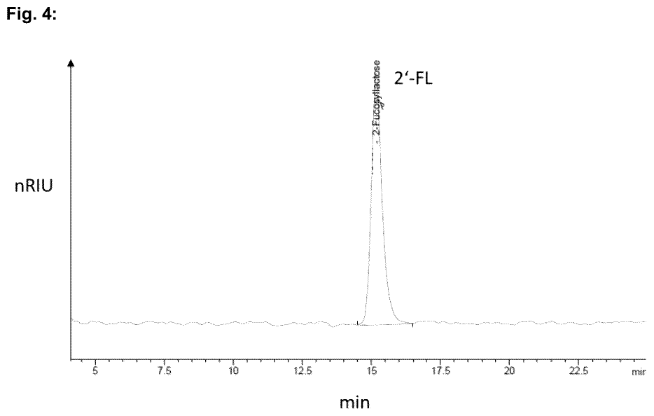

Example 5 shows by way of example a fermentation with complete conversion of lactose (see also FIG. 4)

It is preferable that the method for producing 2′-fucosyllactose is characterized in that at least 4%, more preferably at least 10%, especially preferably at least 25%, and even more preferably at least 50%, more 2′-fucosyllactose is formed with the fusion protein than by the unfused wild-type enzymes of which one domain is included in the fusion protein.

In a preferred embodiment, the method for producing 2′-fucosyllactose is characterized in that at least 47 g/l, preferably at least 53 g/l, and more preferably at least 60 g/l, of 2′-fucosyllactose is formed in the reaction.

Preferably, the method for producing 2′-fucosyllactose is characterized in that less than 1 g/l and more preferably 0 g/l of difucosyllactose is formed in the reaction. This means it is particularly preferable that the formation of DFL is prevented, because in this case the isolation of the 2′-fucosyllactose is much easier and thus economically more efficient, since laborious purification steps to remove DFL can be dispensed with.

In a preferred embodiment, the method for producing 2′-fucosyllactose is characterized in that the expression of the enzyme is induced.

In this case, the promoter controlling the expression of the enzyme of the invention is an inducible promoter, more preferably a promoter inducible by IPTG (isopropyl-ß-D-thiogalactopyranoside).

In this case, the method of the invention has the advantage that synthesis of the product does not commence until the moment of induction, which means that a high cell density is reached first, thereby increasing the yield.

### EXAMPLES

The invention is described in more detail hereinbelow with reference to exemplary embodiments, without being limited thereby.

All molecular biological methods used, such as polymerase chain reaction (PCR), gene synthesis, DNA isolation and purification, DNA modification by restriction enzymes and ligase, transformation, etc., were carried out in the manner known to those skilled in the art, described in the literature or recommended by the respective manufacturers.

### Example 1: Strain Development Based on E. coli K12 for the Production of 2-Fucosyllactose

A strain based on E. coli K12 was developed for the intracellular synthesis of fucosylated HMOs such as 2′-FL. First, the cds for undecaprenyl phosphate glucose phosphotransferase wcaJ was deleted from the genome. The cds for the Ion protease was then removed. The lac operon was modified in that the cds for β-galactosidase (lacZ) and cds for β-galactoside transacetylase (lacA) were deleted, whereas the cds for β-galactoside permease (lacY) was preserved. Lastly, the cds for the cell division inhibitor sulA was deleted.

Deletion of the Cds for wcaJ, Ion and sulA with the Aid of the A-Recombinase According to Datsenko and Wanner (2000, Proc. Natl. Acad. Sci. USA. 97: 6640-5)

For the deletion of wcaJ from the genome of the employed E. coli strain K12, polymerase chain reaction (PCR) using the oligonucleotides wcaJ-del-fw (SEQ ID No. 26) and wcaJ-del-rv (SEQ ID No. 27) and the commercially available plasmid pKD3 (Coli Genetic Stock Center, CGSC: 7631) as matrix was first used to generate a linear DNA fragment that contained a chloramphenicol resistance cassette and that was flanked by approx. 50 base pairs each of the upstream and downstream regions of the wcaJ cds.

In addition, the E. coli strain was transformed with the commercially available plasmid pKD46 (CGSC: 7739) and competent cells then produced according to the particulars of Datsenko and Wanner. These were transformed with the linear DNA fragment generated by PCR. The selection for integration of the chloramphenicol resistance cassette (cat=chloramphenicol acetyltransferase) into the chromosome of E. coli strain K12 was carried out on LB agar plates containing 20 mg/l chloramphenicol. Integration at the correct position in the chromosome was verified by PCR using the oligonucleotides wcaJ-check-fw (SEQ ID No. 28) and wcaJ-check-rv (SEQ ID No. 29) and chromosomal DNA of the chloramphenicol-resistant cells as matrix. This process afforded E. coli cells in which the wcaJ cds had been replaced by the chloramphenicol resistance cassette.

The plasmid pKD46 was then removed again from the cells according to the described procedure (Datsenko and Wanner) and the strain thus generated was designated E. coli K12 wcaJ::cat.

The removal of the chloramphenicol resistance cassette from the chromosome of E. coli strain K12 wcaJ::cat was effected according to the procedure of Datsenko and Wanner with the aid of the plasmid pCP20 (CGSC: 7629), which encodes for the FLP recombinase cds. The chloramphenicol-sensitive wcaJ deletion mutant finally obtained with this method was designated E. coli K12ΔwcaJ.

The deletion of the Ion cds from E. coli strain K12ΔwcaJ was effected using the same method that had previously been used for deletion of the wcaJ cds. However, the generation of the linear DNA fragment with pKD3 (CGSG: 7631) as matrix employed the oligonucleotides Ion-del-fw (SEQ ID No. 30) and Ion-del-rv (SEQ ID No. 31).

The integration of the chloramphenicol resistance cassette into the chromosome of E. coli strain K12ΔwcaJ at the position of the Ion cds was verified by PCR with the oligonucleotides Ion-check-fw (SEQ ID No. 32) and Ion-check-rv (SEQ ID No. 33) and chromosomal DNA of the chloramphenicol-resistant cells.

The removal of the chloramphenicol resistance cassette from the chromosome was again effected as described by Datsenko and Wanner. The resulting strain without the chloramphenicol resistance cassette and characterized by the genomic deletion of the wcaJ cds and Ion cds was designated E. coli K12ΔwcaJΔIon.

The deletion of the sulA cds from E. coli strain K12ΔwcaJΔIon-lac-mod (produced as described in the section “Modification of the lac operon” below) was effected using the same method that had previously been used for deletion of the wcaJ cds. However, for generation of the linear DNA fragment that contained a kanamycin resistance gene and that was flanked by 50 homologous base pairs each of the regions upstream and downstream of the genomic cds of sulA, the oligonucleotides sulA-del-fw (SEQ ID No. 34) and sulA-del-rv (SEQ ID No. 35) and pKD13 (CGSC:7633 GenBank seq. AY048744) were used as matrix.

Selection for the integration of the kanamycin resistance cassette (kanR) into the chromosome of E. coli strain K12ΔwcaJΔIon-lac-mod at the position of the sulA cds was carried out initially on LB agar plates containing 50 mg/l kanamycin. The integration was then verified by PCR with the oligonucleotides sulA-check-fw (SEQ ID No. 36) and sulA-check-rv (SEQ ID No. 37) and chromosomal DNA of kanamycin-resistant cells. The removal of the kanamycin resistance cassette from the chromosome was likewise effected in the same way as the chloramphenicol resistance cassette, according to the procedure of Datsenko and Wanner. The resulting strain after removal of the kanamycin resistance cassette was designated E. coli K12ΔwcaJΔIonΔsulA-lac-mod.

### For the Production of 2′-FL, the Strain was Transformed with the Appropriate Expression Plasmids (See Example 2)

Modification of the lac operon according to a plasmid integration method of Hamilton et al. (1989, J. Bacteriol. 171 (99: 4617-4622)

For the parallel deletion of lacZ and lacA from the lac operon lacZYA, wherein the operon structure with promoter, RBS, and start codon and also the lacY cds were preserved, the homologous recombination method described by Hamilton et al. (1989) was used.

This was done by employing a plurality of PCRs using overlapping oligonucleotides and the genomic DNA of wt E. coli K12 as matrix to generate three linear DNA fragments (PCR1: lac-1-fw+lac-2-rv (SEQ ID Nos. 38, 39), PCR2: lac-3-fw+lac-4-rv (SEQ ID Nos. 40, 41), PCR3: lac-5-fw+lac-6-rv (SEQ ID Nos. 42, 43)), which were then fused on the basis of the overlapping regions by two further polymerase chain reactions. For this, the linear DNA fragments from PCR1 and PCR2 were first fused (PCR4) with the aid of the primers lac-1-fw (SEQ ID No. 38) and lac-4-rv (SEQ ID No. 41), in order to then link the resulting DNA fragment to the DNA fragment from PCR3 and the oligonucleotides lac-7-fw (SEQ ID No. 44) and lac-8-rv (SEQ ID No. 45) (PCR5). The final linear DNA fragment contained a 515 bp homologous region downstream of the lacA cds, the lacY cds, and a 535 bp homologous region upstream of lacZ, the fragment being flanked at each terminus by a BamHI cut site.

For the cloning of the DNA fragment thus obtained into the temperature-sensitive vector pMAK700 (Hamilton et al., 1989, J. Bacteriol. 171 (99: 4617-4622)), the vector and the linear fragment were both treated with the restriction enzyme BamHI and the vector fragment was dephosphorylated with an alkaline phosphatase (rAPid Alkaline Phosphatase, Roche), purified by gel electrophoresis, and then ligated and used for the transformation of competent Stellar E. coli cells (Takara, Shiga-Japan). Selecting for plasmid-containing cells was carried out on the basis of the plasmid-encoded chloramphenicol resistance gene on LB agar with chloramphenicol. Since the plasmid also contains a temperature-sensitive (ts) origin of replication (ori) that results in plasmid replication being possible only at 30° C. but not at 42° C., the cells were incubated at 30° C. To modify the lac operon, E. coli strain K12ΔwcaJΔIon (see above) was transformed at 30° C. with the vector pMAK700-lac-mod. After culturing chloramphenicol-resistant clones in LB medium with chloramphenicol at 30° C., the cultures were plated out on LB agar with chloramphenicol and incubated overnight at 42° C. This allowed the selection of clones that had integrated the complete ts plasmid into the chromosome as a consequence of the flanking homologous regions downstream of lacA and upstream of lacZ and thus can also develop chloramphenicol resistance at elevated temperature. Such clones were isolated and checked for correct plasmid integration by control PCRs with the oligomers pMAK-fw (SEQ ID No. 46) and lac-9-rv (SEQ ID No. 47) or with lac-10-fw (SEQ ID No. 48) and pMAK-rv (SEQ ID No. 49). Because one primer in the plasmid (pMAK-fw/pMAK-rv) and another in the chromosome (lac-9-rv/lac-10-fw) are able to undergo homologous attachment, corresponding linear DNA fragments formed only in the case of correct plasmid integration. The integration strain was designated E. coli K12ΔwcaJΔIon::pMAK700-lac-mod.

To remove the plasmid from the genome, it was necessary for a second recombination to take place. Depending on how this was done, two genomic variants of the strain resulted. In the first case, the plasmid recombined in the same way as in the “in” recombination, resulting in the wild type again. Alternatively, the plasmid recombined such that the altered gene locus remained in the genome and the plasmid with the wild-type gene locus was released. To deintegrate the plasmid from the genome, E. coli 12ΔwcaJΔIon::pMAK700-lac-mod in LB medium with chloramphenicol was incubated for 4 hours at 42° C. in LB medium with chloramphenicol and then in LB medium without chloramphenicol at 30° C. and passaged multiple times. This process resulted in some of the cells being in turn able to effect the “out” recombination of the plasmid from the genome and to lose the plasmid on account of the lack of selection pressure. For the isolation of individual clones, a dilution of the culture was plated out on LB agar and incubated at 30° C. To check whether the plasmid had been lost in the clones, these were then streaked on LB agar with chloramphenicol. Chloramphenicol-sensitive clones were finally checked for the desired genetic modification by PCR with the primers lac-11-fw (SEQ ID No. 50) and lac-12-rv (SEQ ID No. 51) and by sequencing. The resulting strain was designated E. coli K12ΔwcaJΔIon-lac-mod.

### Example 2: Cloning the Cds of the Fucosyltransferases futC, futC*, futL, Hybrids, and Shortened Variants for the Fermentative Production of 2-Fucosyllactose

### Preparation of the Expression Vector:

The expression vector used was pWC1. This is a low-copy plasmid. pWC1 is present in the cells with approx. 10 copies per cell based on the pACYC origin of replication. The plasmid map is shown in FIG. 1 and the sequence disclosed in SEQ ID No. 1, the positions of customary restriction enzymes (with 6-base recognition sequence) being indicated on the plasmid map.

On this plasmid, the coding sequence (cds) for the respective enzyme was placed under the control of the lactose- and IPTG-inducible promoter ptac. The vector contains restriction sites for the enzymes EcoRI and XbaI. Treatment of the plasmid with these enzymes results in the formation inter alia of a large fragment with 4799 bp. This was isolated by agarose gel electrophoresis (QIAquick® Gel Extraction Kit, Quiagen) and treated with alkaline phosphatase (rAPid Alkaline Phosphatase, Roche) to avoid religation. This vector fragment was used for cloning the various fucosyltransferases.

Cloning of the Cds for futC, futC*, and futL

The cds of the fucosyltransferases futC (SEQ ID No. 2) and futC* (SEQ ID No. 6), modified for optimal codon usage of E. coli, were synthesized by GeneArt (Thermo Fisher, Regensburg) and futL (SEQ ID No. 4) by Genewiz (Leipzig). The cds coding for futC and futC* underwent PCR amplification under standard conditions in two separate mixtures with the primer pairs futC/futC*-fw (SEQ ID No. 20) and futC/futC*-rv (SEQ ID No. 21), and the cds coding for futL was amplified in a third mixture with the primer pairs futL-fw (SEQ ID No. 22) and futL-rv (SEQ ID No. 23), which are used for introduction of an EcoRI or XbaI cut site. Because of the substantial homology of the futC cds and futC* cds, it was possible to use one primer pair (futC/futC*-fw and futC/futC*-rv) for both these constructs.

The corresponding PCR products were subsequently likewise treated with the restriction enzymes EcoRI and XbaI and then each combined with the enriched dephosphorylated vector fragment in a ligase mixture. The ligation mixture was then transformed into competent Stellar E. coli cells (Takara, Shiga-Japan) using standard methods. Single colonies with successfully ligated plasmid were selected by means of tetracycline resistance. Some plasmids from these colonies were analyzed by restriction pattern and sequencing. Finally, the correct plasmids were used for the production experiments or for further cloning. The resulting plasmids were pWC1-futC, pWC1-futC*, and pWC1-futL.

Introduction of the Scal Restriction Cut Site into the futL Cds:

First, the entire futL expression plasmid was amplified by PCR. The primers here contained a new restriction cut site (Scal) in the cds of futL (SEQ ID No. 4). The aim was to introduce a restriction cut site into the linker sequence between the two enzyme domains of the fucosyltransferase without altering the amino acid sequence. It was then possible to exchange the two domains for any desired alternative domains by means of the three restriction sites (EcoRI, Scal, and XbaI). For the PCR reaction, the expression vector pWC1-futL described above served as matrix; the primers used were futL-Sca-fw (SEQ ID No. 52) and futL-Sca-rv (SEQ ID No. 53).

At the end of the PCR reaction, the plasmid DNA was chromatographically purified before adding the restriction enzyme DpnI (10 units, NEB) to remove the methylated matrix DNA from the mixture. The DpnI mixture was incubated at 37° C. for 1 hour. This was followed by chromatographic purification of the DNA (Macherey & Nagel: NucleoSpin® Gel and PCR Clean-up-Kit) and transformation into competent Stellar E. coli cells (Takara, Shiga-Japan). Selecting for positive clones was carried out as described above. The vector was designated pWC1-futL(Scal).

Cloning of the Cds of the Fusion Proteins futL/futC*, futC*/futL, and futC/futL:

For the cloning of the hybrid enzymes, the plasmid pWC1-futL(Scal) was treated with the restriction enzymes Scal and XbaI. The approx. 5243 bp vector backbone fragment contains the N-terminal portion of the futL cds (SEQ ID No. 4). This fragment was dephosphorylated and enriched by agarose gel electrophoresis.

In parallel thereto, a PCR was carried out with the primers C-futC*-fw and C-futC*-rv (SEQ ID Nos. 54, 55) and the vector pWC-1-futC* as matrix. The PCR product consisted mainly of the C-terminal domain of futC* (SEQ ID No. 6).

At the end of the PCR reaction, the DNA was treated with the restriction enzymes Scal and XbaI, chromatographically purified, and then used together with the plasmid fragment in a ligation mixture. The ligation mixture was then transformed into competent Stellar E. coli cells (Takara, Shiga-Japan) using standard methods. Single colonies with successfully ligated plasmid were selected by means of tetracycline resistance. Some plasmids from these colonies were analyzed by restriction pattern and sequencing. Finally, the correct plasmids were used for the production experiments or for further cloning.

The resulting plasmid was designated pWC1-futL/futC*.

Similarly, for the creation of the futC*/futL hybrid (SEQ ID No. 8 (DNA)/SEQ ID No. 9 (PRT)) the vector pWC1-futL(Scal) was prepared by treatment with the restriction enzymes EcoRI and Scal. The approx. 5240 bp vector fragment here contained the C-terminal domain of the futL cds (SEQ ID No. 4).

By analogy with the previous hybrid cloning, the N-terminal domain of the futC* cds (SEQ ID No. 6) was amplified by PCR. The vector pWC1-futC* again served as template and the primer pair used was (N-futC*-fw (SEQ ID No. 56) and N-futC*-rv (SEQ ID No. 57).

The vector fragment was after dephosphorylation and enrichment ligated together with the PCR product that had been treated with restriction enzyme (EcoRI/Scal) and likewise enriched, and the mixture was transformed into competent Stellar E. coli cells (Takara, Shiga-Japan) using standard methods. The desired hybrid plasmid was isolated as described above. The resulting plasmid was designated pWC1-futC*/futL.

Similarly, the hybrid construct futC/futL (SEQ ID No. 12 (DNA)/SEQ ID No. 13 (PRT)) was cloned from the vector pWC1-futL(Scal).

As described above, the vector fragment was generated with the C-terminal portion of the futL cds and a PCR product was generated as described below and the two were treated further and ligated as described above. The matrix used for the PCR was the vector pWC1-futC, which contains the cds for futC (SEQ ID No. 2), and the primer pair used was N-futC*-fw (SEQ ID No. 56) and N-futC*-rv (SEQ ID No. 57).

The resulting plasmid was designated pWC1-futC/futL.

Cloning of the Fucosyltransferase Expression Plasmids with rcsA

To increase the de-novo synthesis of activated fucose (GDP-fucose) in E. coli, an optimized Shine-Dalgarno sequence (AGGAGGU; SDS), followed by the E. coli endogenous cds for RcsA (SEQ ID No. 18), was cloned directly C-terminally of the cds of the respective fucosyltransferase in an operon with the fucosyltransferase. For this, the primers rcsA-fw (SEQ ID Nr. 24) and resA-rv (SEQ ID No. 25), which had been used to introduce a NheI or XbaI cut site, were used to amplify cds from rcsA. Genomic DNA of E. coli K12 served as the matrix.

By analogy with the cloning of the fucosyltransferase expression vectors, the latter, i.e. pWC1-futL, pWC1-futC, pWC1-futC*, pWC1-futL/futC*, pWC1-futC*/futL, and pWC1-futC/futL, were each treated with the restriction enzyme XbaI, dephosphorylated, and enriched by agarose gel electrophoresis. The rcsA PCR product was treated with the restriction enzymes NheI and XbaI. This was followed by chromatographic purification of the DNA (Macherey & Nagel: NucleoSpin®) Gel and PCR Clean-up-Kit). For the ligation mixture, the respective enriched dephosphorylated vector fragment was combined with the enriched PCR product. The ligation mixture was then transformed into competent Stellar E. coli cells (Takara, Shiga-Japan) using standard methods. Single colonies with successfully ligated plasmid were selected by means of tetracycline resistance. Some plasmids from these colonies were analyzed by restriction pattern and sequencing. Finally, the correct plasmids (pWC1-futC*-rcsA, pWC1-futC-rcsA, pWC1-futL-rcsA, pWC1-futC*/futL-rcsA, pWC1-futL/futC*-rcsA, pWC1-futC/futL-rcsA) were used for production experiments or for further clonings.

Cloning of Shortened futC*/futL Variants:

For the cloning of the futC*/futL variant futC*/futL(Δ8aa) shortened by 8 aa, in separate PCRs the cds of the futC*/futL hybrid (SEQ ID No. 8) based on pWC1-fuc*/futL was first amplified with the primers futC*-short-fw (SEQ ID No. 58) and futC*-short8-rv (SEQ ID No. 59) and the cds for rcsA (SEQ ID Nr. 18) based on pWC1-futC-rcsA amplified with the primers rcsA-2-fw (SEQ ID No. 60) and rcsA-2-rv (SEQ ID No. 61). The use of the terminally homologous oligonucleotides futC*-short8-rv (SEQ ID No. 59) and rcsA-2-fw (SEQ ID No. 60) then allowed the fusion of the resulting linear DNA fragments in a further PCR with the primers futC*-short-fw (SEQ ID No. 58) and rcsA-2-rv (SEQ ID No. 61). The final linear DNA fragment contained an EcoRI cut site, the cds for futC*/futL(Δ8aa) (SEQ ID No. 14), an RBS, the cds for rcsA, and an XbaI cut site.

The linear DNA fragment for the futC*/futL variant futC*/futL(Δ15aa) shortened by 15 aa was cloned in the same way, but with the oligonucleotides futC*-short15-rv (SEQ ID No. 62) instead of futC*-short8-rv (SEQ ID No. 59) and rcsA-3-fw (SEQ ID No. 63) instead of resA-2-fw (SEQ ID No. 60). The final linear DNA fragment contained an EcoRI cut site, the cds for futC*/futL(Δ15aa) (SEQ ID No. 16), an RBS, the cds for rcsA, and an XbaI cut site.

Finally, both linear DNA fragments were treated with EcoRI and XbaI and then each ligated with the enriched dephosphorylated vector fragment (pWC1 cut with EcoRI and XbaI, see above) and transformed for transformation of competent Stellar E. coli cells (Takara, Shiga-Japan). Single colonies with ligated plasmids were selected on the basis of the introduced tetracycline resistance. Before being used in 2′-FL production experiments to demonstrate fucosyltransferase activity, the plasmids were analyzed by restriction pattern and sequencing. The plasmids pWC1-futC*/futL(Δ8aa)-rcsA and pWC1-futC*/futL(Δ15aa)-rcsA were obtained.

### Example 3: Influence of the Different Fucosyllactose Transferases on the Fermentative Production of 2-Fucosyllactose and Difucosyllactose in the 1 L Fermenter

30 mL of LB medium (3% peptone, 0.5% yeast extract, 0.5% NaCl) in a 300 mL baffled conical flask was inoculated, using an inoculation loop, from a densely covered LB agar plate on which had previously been plated out a single clone of the production strain E. coli K12ΔwcaJΔIonΔsulA-lac-mod from example 1 transformed with the respective production plasmid from example 2 (pWC1-futL-rcsA, pWC1-futC-rcsA, pWC1-futC*-rcsA, pWC1-futC/futL-rcsA, pWC1-futC*/futL-rcsA, pWC1-futL/futC*-rcsA, pWC1-futC*/futL(Δ8aa)-rcsA, pWC1-futC*/futL(Δ15aa)-rcsA). After incubating for 4.5-5 hours in the bacteria shaker (145 rpm, 30° C.), the OD₆₀₀ was between 1.5 and 3.0 (OD₆₀₀ refers to the spectrophotometrically determined optical density at 600 nm). For the fermentation in Biostat β-DCU research fermenters from Sartorius, 6-13 ml of the precultures was in each case transferred to the initially charged medium in the fermenter. The initial volume after inoculation was approx. 1 L.

The fermentation medium contained the following components: 1 g/l of NaCl, 150 mg/l of FeSO₄·7H₂O, 2 g/l of trisodium citrate dihydrate, 10 g/l of KH₂PO₄, 5 g/l of (NH₄)₂SO₄, 1.5 g/l of HighExpress II (Kerry), 1.0 g/l of Amisoy (Kerry), 0.5 g/l of Hy-Yeast 412 (Kerry), and 10 ml/l of trace element solution (the fermenter was initially charged with a solution of these components in H₂O and this was autoclaved at 121° C. for 20 min). The trace element solution was composed of 150 mg/l of Na₂MoO₄·2H₂O, 300 mg/l of H₃BO₃, 200 mg/l of CoCl₂·6H₂O, 250 mg/l of CuSO₄·5H₂O, 1.6 g/l of MnCl₂·4H₂O, and 1.35 g/l of ZnSO₄·7H₂O. The pH of the medium was adjusted to 6.8 by pumping in a 25% NH₄OH solution. To this was then added under sterile conditions 15 g/l of glucose, 1.2 g/l of MgSO₄·7H₂O, 225 mg/l of CaCl·2H₂O, 5 mg/l of vitamin B1, and 20 mg/l of tetracycline from adequate stock solutions, after which the inoculum was transferred from the shaker flask to the fermenter.

During the fermentation, the culture was stirred at 400-1500 rpm and aerated with a constant 2 slpm of air supplied via a sterilizing microbial filter. The oxygen partial pressure was maintained at 50% by adjusting the stirring speed; in the late exponential phase it was necessary to enrich the supply air with pure O₂ to an O₂ content of 32%, in order to ensure the desired nominal value of 50% for the O₂ partial pressure in the culture solution. The pH was maintained at 6.8 by automatic correction with 25% NH₄OH solution or 20% H₃PO₄ solution. The temperature was initially 30° C. and 30 min before induction was gradually reduced from 30° C. to 25° C. over a 30 min period. The temperature was then maintained at 25° C. until the end of the fermentation (65 h). Excessive foaming was prevented by the automatically controlled addition of defoamer (Struktol J673, Schill & Seilnacher, 10% (v/v) in H₂O).

Glucose and lactose were added, depending on the phase in the fermentation, via two separate (sterile) feed solutions. The glucose content was determined with the aid of a glucose analyzer from YSI. In the first phase from inoculation, the glucose in the initially charged medium was consumed. In a second phase starting approx. 10 h after the start of the fermentation, at a glucose concentration of 0 g/l, the culture was continuously fed a 60% (w/w) glucose feed solution with additives (660.2 g/kg of glucose monohydrate (Biesterfeld-Spezialchemie), 2.5 g/kg of vitamin B1 from a 5 g/l stock solution, 3.65 g/kg of CaCl₂·2H₂O from a 147 g/l stock solution, 12.31 g/kg of MgSO₄·7H₂O from a 240 g/l stock solution, 4.04 g/kg of trace element solution (see above), and 317.3 g/kg of demineralized water), with the aim of providing the culture with an unlimited glucose supply. In a third phase characterized by complete consumption of the continuous glucose feed, the continuous addition of the glucose feed was, approx. 18.5 h after inoculation, reduced to a constant 9.2 g/L/h until the end of the fermentation (65 h) and expression of the respective 1,2-fucosyltransferase and rcsA induced by adding 0.25 mM of IPTG. In parallel thereto, at the beginning of the third phase, 20 g/l of lactose was added as a batch from a 25% (w/w) lactose solution (263 g/kg of α-D-lactose monohydrate (abcr) dissolved in 737 g/kg of demineralized H₂O and then continuous feeding with 4 g/l/h of the 25% (w/w) lactose solution maintained until the end of the fermentation. A total of 65 g/l of lactose was thus added based on the starting volume of 1 L.

In an alternative mixture (mixture 2) for the fermentative production of 2′-FL, the temperature was 30 min before the start of induction gradually reduced to 27° C. over a 30-min period and at induction 30 g/l of lactose was added as a batch and continuous feeding with 5 g/l/h of a 25% (w/w) lactose solution maintained until the end of the fermentation. This corresponded to a total of 86 g/l of lactose based on the initial volume of 1 L.

The 2′-FL, 3′-FL, DFL, and lactose contents of the medium after 65 h was determined chromatographically from the cell-free supernatant of the sample as described in example 4 and summarized in g/l in Table 1.

TABLE 1
Comparison of the different fucosyltransferase
activities in terms of 2′-FL and DFL yields.

| | | |
|---|---|---|
| Fucosyltransferase expressed in the | 2′-FL | DFL |
| fermentation | (g/l) | (g/l)/ |

Mixture 1: Fermentation conditions 25° C., 65 g/l lactose

| | | |
|---|---|---|
| futC (SEQ ID No. 3) | 34 | 17 |
| futC* (SEQ ID No. 7) | 20 | 15 |
| futL (SEQ ID No. 5) | 45 | — |
| futL/futC* (SEQ ID No. 11) | 16 | 15 |
| futC*/futL (SEQ ID No. 9) | 53 | — |
| futC/futL (SEQ ID No. 13) | 47 | — |

Mixture 2: Fermentation conditions 27° C., 86 g/l lactose

| | | |
|---|---|---|
| futC*/futL (SEQ ID No. 9) | 60 | — |
| futC*/futL(Δ8aa) (SEQ ID No. 15) | 55 | — |
| futC*/futL(Δ15aa) (SEQ ID No. 17) | — | — |
| futC/futL (SEQ ID No. 13) | 51 | — |

### Example 4: HPLC Analysis of the Fermentative Production of 2-Fucosyllactose

For the chromatographic determination of the 2′-FL, 3′-FL, DFL, and lactose contents in the medium, 1 ml of the culture broth was after fermentation for 65 h centrifuged for 5 min at 13000 rpm in a benchtop centrifuge and the clear supernatant diluted 1:2 to 1:5 in demineralized water. 300 μl of the dilution was then filtered into an HPLC reservoir vessel using a 0.2 μm syringe filter.

For separation of the analytes, a TSKgel amide-80 column (Tosoh Bioscience, 250 mm×4.6 mm; particle size 5 μm) and a corresponding guard column (TSKgel Guardgel amide-80, Tosoh Bioscience, 15 mm×3.2 mm) were used in an Agilent 1200/1260 HPLC system having the following modules: binary pump, degasser, autosampler, thermostated column oven, 1260 RI detector. The temperature of the column oven was 30° C. The eluent used was a degassed mixture of H₂O (30%) and acetonitrile (70%).

After equilibration of the column, 10 μl of the prepared sample was each time injected from an autosampler cooled to 15° C. Elution was then carried out isocratically at a flow rate of 1 ml/min for 25 min. An RI detector (temp. 35° C.) and a Q-TOF Impact II mass spectrometer from Bruker Daltonics were used for detection.

Peaks were assigned to the analytes on the basis of the retention times of standard solutions (2′-FL: 15.4 min, 3′-FL: 17.3 min; DFL: 22.3 min; lactose: 12.2 min). The concentration of the analytes in g/l was finally determined through integration of the peak areas and with the aid of calibration lines for the standards, taking into account the respective dilution.

### Example 5: Complete Fucosylation of Lactose to 2′-Fucosyllactose without Formation of Difucosyllactose in the 1 L Fermenter

As previously described in example 3, mixture 2, the production strain E. coli K12ΔwcaJΔIonΔsulA-lac-mod with a production plasmid coding for a fusion protein homologous to futC*/futL was transformed and fermented. In a departure from example 3, the lactose feed was ended after 65 h, whereas continuous addition of glucose was maintained. After 88 h the fermentation was ended and, as before, the sugars present in the culture supernatant were determined by HPLC. The formation of 67 g/l of 2′-FL alongside 0 g/l DFL and 0 g/l residual lactose showed that the fusion protein is able to achieve complete conversion of lactose into 2′-FL without formation of the by-product DFL, which means that no process steps to remove lactose and difucosyllactose are needed during subsequent workup.

### Abbreviations

aa: Amino acid(s)
ΔZaa: Deletion of Z amino acids, where Z indicates the number of deleted amino acids
OD₈₀₀: Optical density at a wavelength of 600 nm
RBS: Ribosome binding site
FT: Fucosyltransferase
GT: Glycosyltransferase
nRIU: Nano refractive index units

The figures show:

FIG. 1: Vector map for the expression plasmid pWC1

Overview of the individual elements of the expression vector. The restriction sites for the employed enzymes EcoRI und XbaI are also marked.

FIG. 2: E. coli strain K12 for the production of 2-fucosyllactose.

For the enzymatic synthesis of 2′-fucosyllactose with the aid of a (specific)) 1,2-fucosyltransferase, an E. coli strain K12 was genetically modified such that it accepts the substrate lactose through the transporter lacY, but is unable to metabolize it. For this, the cds for lacA and lacZ were deleted from the genome. For enhanced intracellular production of GDP-fucose from glucose on the de-novo synthesis pathway, the cds for the Ion protease was deleted from the genome in order to prevent proteolytic breakdown of the transcription activator resA for the genes of this particular de-novo synthesis pathway. The level of rcsA can additionally be increased through overexpression of a plasmid. The deletion of wcaJ prevents consumption of GDP-fucose for the synthesis of colonic acids, with the result that the activated fucose can be transferred by a plasmid-encoded 1,2-fucosyltransferase to the internalized lactose, the expression of a specific 1,2-fucosyltransferase preventing the formation of the undesired by-product difucosyllactose (DFL) through fucosylation of 2′-fucosyllactose.

FIG. 3: HPLC analysis of the synthesis of 2′-FL and DFL.

The HPLC analysis of the supernatants after the fermentation shows the production of 2′-FL and—where present—DFL using FutC*/FutL, FutC, and FutL. The chromatograms of the 2′-FL, lactose, and DFL standards are shown for comparison.

FIG. 4: HPLC analysis of the synthesis of 2′-FL with complete consumption of lactose.

The HPLC analysis of the supernatants after the fermentation shows the production of 2′-FL and—where present—DFL, and also the residual lactose, using an enzyme homologous to FutC*/FutL.

1-15. (canceled)

16. An enzyme, characterized in that it is a fusion protein,
i) that comprises as an N-terminal domain at least amino acids 1-129 of SEQ ID No. 7 or an amino acid sequence at least 80% identical thereto and
ii) that comprises at least amino acids 155-286 of SEQ ID No. 5 or an amino acid sequence at least 80% identical thereto as a C-terminal domain and has fucosyltransferase activity,
the N-terminal domain and the C-terminal domain being derived from two different fucosyltransferases.

17. The enzyme as claimed in claim 16, wherein the amino acid sequences of the N-terminal and C-terminal domain of the fusion protein are microbial sequences or a sequence homologous thereto.

18. The enzyme as claimed in claim 16, wherein the amino acid sequences of the N-terminal and C-terminal domain of the fusion protein are sequences of the genus Helicobacter or a sequence homologous thereto.

19. The enzyme as claimed in claim 16, wherein the amino acid sequence of the N-terminal domain of the fusion protein is amino acids 1-148 of SEQ ID No. 7 or an amino acid sequence at least 80% identical thereto.

20. The enzyme as claimed in claim 16, wherein the C-terminal domain comprises at least amino acids 155-286 of SEQ ID No. 5 or an amino acid sequence at least 80% identical thereto.

21. The enzyme as claimed in claim 16, wherein the amino acid sequence of the C-terminal domain of the fusion protein is amino acids 142-286 of SEQ ID No. 5 or an amino acid sequence at least 80% identical thereto.

22. The enzyme as claimed in claim 16, wherein the fusion protein is SEQ ID No. 9, SEQ ID No. 13, SEQ ID No. 15 or an amino acid sequence at least 80% identical thereto.

23. A method for producing 2′-fucosyllactose, wherein lactose is in a reaction mixture in the presence of at least one substance selected from the group of substances consisting of glucose, glycerol, sucrose, fucose, and GDP-, ADP-, CDP-, and TDP-fucose reacted with at least one enzyme as claimed in claim 16.

24. The method as claimed in claim 23, wherein lactose undergoes complete conversion without more than 5% of DFL being formed.

25. The method as claimed in claim 23, wherein the reaction mixture is a culture of microorganisms that recombinantly express the enzyme.

26. The method as claimed in claim 25, wherein 2′-fucosyllactose is isolated from the culture supernatant.

27. The method as claimed in claim 23, wherein at least 4% more 2′-fucosyllactose is formed by the fusion protein than by the unfused wild-type enzymes of which one domain is included in the fusion protein.

28. The method as claimed in claim 23, wherein at least 47 g/l of 2′-fucosyllactose is formed in the reaction.

29. The method as claimed in claim 23, wherein less than 1 g/l of difucosyllactose is formed in the reaction.


SEQUENCE LISTING (63 sequences)

SEQ ID NO: 1 - DNA, 4846 bp, E.coli
  pWC1 (1)..(4846)
ttgacaattaatcatcggctcgtataatgtgtggaattgtgagcggataacaatttcacacaggaaacagaattctaaggaggaaattatgcagaattcaattggtacccgggagatctagaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcggccgcatatgagctctccggaagatctctcgaggtcgacgtacgtgcccatggatgcatttaattaattcgaagttaacttaagcttgtacactagtaaagttctgctatgtggcgcggtattatcccgtgttgacgccgggcaagagcaactcggtcgccgcatacactattctcagaatgacttggttgagtccatggcggccgccttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagctagatttcagtgcaatttatctcttcaaatgtagcacctgaagtcagccccatacgatataagttgtaattctcatgtttgacagcttatcatcgataagctccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatggcaggcctcccccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctg

SEQ ID NO: 2 - DNA, 903 bp, Helicobacter pylori
  futC (1)..(903)
atggcctttaaagttgttcagatttgtggtggtctgggcaatcagatgtttcagtatgcatttgcaaaaagcctgcaaaagcatctgaatacaccggttctgctggataccaccagttttgattggagcaatcgtaaaatgcagctggaactgtttccgattgatctgccgtatgcaaatgccaaagaaattgccattgccaagatgcagcatctgccgaaactggttcgtgatgcactgaaatacattggttttgatcgtgtgagccaagagatcgtgtttgaatatgaaccgaaactgctgaaaccgagccgtctgacctattttttcggttattttcaagatccgcgttactttgatgcaattagcagcctgattaaacagacctttacactgcctccgcctccggaaaataacaaaaacaacaacaaaaaagaggaagaatatcagcgcaaactgagcctgattctggcagcaaaaaatagcgtttttgtgcatattcgtcgcggtgattatgttggtattggttgtcagctgggtatcgactatcagaaaaaagcactggaatatatggcaaaacgtgtgccgaatatggaactttttgttttttgcgaggacctgaaattcacccagaatctggatctgggttatccgtttaccgatatgaccacacgtgataaagaagaagaggcatattgggatatgctgctgatgcagagctgtaaacatggtattattgccaacagcacctatagttggtgggcagcctatctgatggaaaatccggaaaaaatcattatcggtccgaaacattggctgtttggccatgaaaacattctgtgtaaagaatgggtgaaaatcgaaagccactttgaagtgaaaagccagaaatataacgcctaa

SEQ ID NO: 3 - protein, 300 aa, Helicobacter pylori
  FutC (1)..(300)
MAFKVVQICGGLGNQMFQYAFAKSLQKHLNTPVLLDTTSFDWSNRKMQLELFPIDLPYANAKEIAIAKMQHLPKLVRDALKYIGFDRVSQEIVFEYEPKLLKPSRLTYFFGYFQDPRYFDAISSLIKQTFTLPPPPENNKNNNKKEEEYQRKLSLILAAKNSVFVHIRRGDYVGIGCQLGIDYQKKALEYMAKRVPNMELFVFCEDLKFTQNLDLGYPFTDMTTRDKEEEAYWDMLLMQSCKHGIIANSTYSWWAAYLMENPEKIIIGPKHWLFGHENILCKEWVKIESHFEVKSQKYNA

SEQ ID NO: 4 - DNA, 861 bp, Helicobacter mustelae
  futL (1)..(861)
atggattttaagatcgtgcaagttcacggtggtctgggcaaccagatgttccagtatgccttcgccaagtctttacaaacccacttaaatattccggtgctgctggataccacttggttcgattatggcaaccgcgaactgggtctgcatctgttcccgattgatttacaatgcgccagcgcacagcagatcgcagccgcacatatgcagaatctgccgcgcttagtgcgcggtgcattacgccgcatgggtttaggtcgcgttagcaaagaaatcgtgtttgagtatatgcccgaactgttcgagccgagccgcattgcctatttccacggctacttccaagatcctcgctatttcgaggacattagcccgctgattaagcagacctttactttacctcatccgacagagcacgcagagcagtatagccgtaaactgagccagattttagccgccaagaacagtgtgttcgtgcacattcgtcgcggcgactatatgcgtttaggctggcaattagacattagctatcaattacgcgctattgcttacatggccaagcgtgttcagaatctggagctgtttctgttctgcgaggatttagagtttgtgcagaatttagatttaggttatccgttcgtggacatgaccacacgcgatggtgccgcccactgggacatgatgctgatgcagagctgcaaacatggcatcatcaccaacagcacctatagttggtgggccgcctatttaatcaaaaatccggagaagatcatcatcggcccgagccattggatttacggcaacgagaatattctgtgtaaagattgggttaaaattgaaagccagtttgagaccaaaagttaa

SEQ ID NO: 5 - protein, 286 aa, Helicobacter mustelae
  FutL (1)..(286)
MDFKIVQVHGGLGNQMFQYAFAKSLQTHLNIPVLLDTTWFDYGNRELGLHLFPIDLQCASAQQIAAAHMQNLPRLVRGALRRMGLGRVSKEIVFEYMPELFEPSRIAYFHGYFQDPRYFEDISPLIKQTFTLPHPTEHAEQYSRKLSQILAAKNSVFVHIRRGDYMRLGWQLDISYQLRAIAYMAKRVQNLELFLFCEDLEFVQNLDLGYPFVDMTTRDGAAHWDMMLMQSCKHGIITNSTYSWWAAYLIKNPEKIIIGPSHWIYGNENILCKDWVKIESQFETKS

SEQ ID NO: 6 - DNA, 903 bp, artificial
  Consensus sequence
  futC* (1)..(903)
atggcctttaaagttgttcagatttgtggtggtctgggcaatcagatgtttcagtatgcatttgcaaaaagcctgcaaaagcatctgaatacaccggttctgctggatattaccagctttgattggagcaatcgtaaaatgcagctggaactgtttccgattgatctgccgtatgcaagcgcaaaagaaattgccattgcaaagatgcagcatctgccgaaactggttcgtgatgcactgaaatgtatgggttttgatcgtgtgagccaagaaatcgtgtttgaatatgaaccgaaactgctgaaaccgagccgtctgacctattttttcggttattttcaagatccgcgttacttcgatgcaattagtccgctgattaaacagacctttacactgcctccgcctccggaaaataacaaaaacaacaacaaaaaagaggaagagtaccaccgtaaactgagcctgattctggcagcaaaaaatagcgtttttgtgcatattcgtcgcggtgattatgttggtattggttgtcagctgggtatcgactatcagaaaaaagcactggaatatatggcaaaacgtgtgccgaatatggaactttttgttttttgtgaggacctggaatttacccagaatctggatctgggctatccgtttatggatatgaccacacgtgataaagaagaagaggcatattgggatatgctgctgatgcagagctgtcagcatggtattattgcaaatagcacctatagttggtgggcagcctatctgattaataacccggaaaaaatcatcatcggtccgaaacattggctgtttggccatgaaaacattctgtgtaaagaatgggtgaaaatcgaaagccactttgaagtgaaaagccagaaatataacgcctaa

SEQ ID NO: 7 - protein, 300 aa, artificial
  Consensus sequence
  FutC* (1)..(300)
MAFKVVQICGGLGNQMFQYAFAKSLQKHLNTPVLLDITSFDWSNRKMQLELFPIDLPYASAKEIAIAKMQHLPKLVRDALKCMGFDRVSQEIVFEYEPKLLKPSRLTYFFGYFQDPRYFDAISPLIKQTFTLPPPPENNKNNNKKEEEYHRKLSLILAAKNSVFVHIRRGDYVGIGCQLGIDYQKKALEYMAKRVPNMELFVFCEDLEFTQNLDLGYPFMDMTTRDKEEEAYWDMLLMQSCQHGIIANSTYSWWAAYLINNPEKIIIGPKHWLFGHENILCKEWVKIESHFEVKSQKYNA

SEQ ID NO: 8 - DNA, 882 bp, artificial
  Hybrid sequence of two organisms
  futC*/futL-Hybrid (1)..(882)
atggcctttaaagttgttcagatttgtggtggtctgggcaatcagatgtttcagtatgcatttgcaaaaagcctgcaaaagcatctgaatacaccggttctgctggatattaccagctttgattggagcaatcgtaaaatgcagctggaactgtttccgattgatctgccgtatgcaagcgcaaaagaaattgccattgcaaagatgcagcatctgccgaaactggttcgtgatgcactgaaatgtatgggttttgatcgtgtgagccaagaaatcgtgtttgaatatgaaccgaaactgctgaaaccgagccgtctgacctattttttcggttattttcaagatccgcgttacttcgatgcaattagtccgctgattaaacagacctttacactgcctccgcctccggaaaataacaaaaacaacaacaaaaaagaggaagagtactcacgtaaactgagccagattttagccgccaagaacagtgtgttcgtgcacattcgtcgcggcgactatatgcgtttaggctggcaattagacattagctatcaattacgcgctattgcttacatggccaagcgtgttcagaatctggagctgtttctgttctgcgaggatttagagtttgtgcagaatttagatttaggttatccgttcgtggacatgaccacacgcgatggtgccgcccactgggacatgatgctgatgcagagctgcaaacatggcatcatcaccaacagcacctatagttggtgggccgcctatttaatcaaaaatccggagaagatcatcatcggcccgagccattggatttacggcaacgagaatattctgtgtaaagattgggttaaaattgaaagccagtttgagaccaaaagttaa

SEQ ID NO: 9 - protein, 293 aa, artificial
  Hybrid sequence of two organisms
  FutC*/FutL-Hybrid (1)..(293)
MAFKVVQICGGLGNQMFQYAFAKSLQKHLNTPVLLDITSFDWSNRKMQLELFPIDLPYASAKEIAIAKMQHLPKLVRDALKCMGFDRVSQEIVFEYEPKLLKPSRLTYFFGYFQDPRYFDAISPLIKQTFTLPPPPENNKNNNKKEEEYSRKLSQILAAKNSVFVHIRRGDYMRLGWQLDISYQLRAIAYMAKRVQNLELFLFCEDLEFVQNLDLGYPFVDMTTRDGAAHWDMMLMQSCKHGIITNSTYSWWAAYLIKNPEKIIIGPSHWIYGNENILCKDWVKIESQFETKS

SEQ ID NO: 10 - DNA, 882 bp, artificial
  Hybrid sequence of two organisms
  futL/futC*-Hybrid (1)..(882)
atggattttaagatcgtgcaagttcacggtggtctgggcaaccagatgttccagtatgccttcgccaagtctttacaaacccacttaaatattccggtgctgctggataccacttggttcgattatggcaaccgcgaactgggtctgcatctgttcccgattgatttacaatgcgccagcgcacagcagatcgcagccgcacatatgcagaatctgccgcgcttagtgcgcggtgcattacgccgcatgggtttaggtcgcgttagcaaagaaatcgtgtttgagtatatgcccgaactgttcgagccgagccgcattgcctatttccacggctacttccaagatcctcgctatttcgaggacattagcccgctgattaagcagacctttactttacctcatccgacagagcacgcagagcagtactcacgtaaactgagcctgattctggcagcaaaaaatagcgtttttgtgcatattcgtcgcggtgattatgttggtattggttgtcagctgggtatcgactatcagaaaaaagcactggaatatatggcaaaacgtgtgccgaatatggaactttttgttttttgtgaggacctggaatttacccagaatctggatctgggctatccgtttatggatatgaccacacgtgataaagaagaagaggcatattgggatatgctgctgatgcagagctgtcagcatggtattattgcaaatagcacctatagttggtgggcagcctatctgattaataacccggaaaaaatcatcatcggtccgaaacattggctgtttggccatgaaaacattctgtgtaaagaatgggtgaaaatcgaaagccactttgaagtgaaaagccagaaatataacgcctaa

SEQ ID NO: 11 - protein, 293 aa, artificial
  Hybrid sequence of two organisms
  FutL/FutC*-Hybrid (1)..(293)
MDFKIVQVHGGLGNQMFQYAFAKSLQTHLNIPVLLDTTWFDYGNRELGLHLFPIDLQCASAQQIAAAHMQNLPRLVRGALRRMGLGRVSKEIVFEYMPELFEPSRIAYFHGYFQDPRYFEDISPLIKQTFTLPHPTEHAEQYSRKLSLILAAKNSVFVHIRRGDYVGIGCQLGIDYQKKALEYMAKRVPNMELFVFCEDLEFTQNLDLGYPFMDMTTRDKEEEAYWDMLLMQSCQHGIIANSTYSWWAAYLINNPEKIIIGPKHWLFGHENILCKEWVKIESHFEVKSQKYNA

SEQ ID NO: 12 - DNA, 882 bp, artificial
  Hybrid sequence of two organisms
  futC/futL-Hybrid (1)..(882)
atggcctttaaagttgttcagatttgtggtggtctgggcaatcagatgtttcagtatgcatttgcaaaaagcctgcaaaagcatctgaatacaccggttctgctggataccaccagttttgattggagcaatcgtaaaatgcagctggaactgtttccgattgatctgccgtatgcaaatgccaaagaaattgccattgccaagatgcagcatctgccgaaactggttcgtgatgcactgaaatacattggttttgatcgtgtgagccaagagatcgtgtttgaatatgaaccgaaactgctgaaaccgagccgtctgacctattttttcggttattttcaagatccgcgttactttgatgcaattagcagcctgattaaacagacctttacactgcctccgcctccggaaaataacaaaaacaacaacaaaaaagaggaagagtactcacgtaaactgagccagattttagccgccaagaacagtgtgttcgtgcacattcgtcgcggcgactatatgcgtttaggctggcaattagacattagctatcaattacgcgctattgcttacatggccaagcgtgttcagaatctggagctgtttctgttctgcgaggatttagagtttgtgcagaatttagatttaggttatccgttcgtggacatgaccacacgcgatggtgccgcccactgggacatgatgctgatgcagagctgcaaacatggcatcatcaccaacagcacctatagttggtgggccgcctatttaatcaaaaatccggagaagatcatcatcggcccgagccattggatttacggcaacgagaatattctgtgtaaagattgggttaaaattgaaagccagtttgagaccaaaagttaa

SEQ ID NO: 13 - protein, 293 aa, artificial
  Hybrid sequence of two organisms
  FutC/FutL-Hybrid (1)..(293)
MAFKVVQICGGLGNQMFQYAFAKSLQKHLNTPVLLDTTSFDWSNRKMQLELFPIDLPYANAKEIAIAKMQHLPKLVRDALKYIGFDRVSQEIVFEYEPKLLKPSRLTYFFGYFQDPRYFDAISSLIKQTFTLPPPPENNKNNNKKEEEYSRKLSQILAAKNSVFVHIRRGDYMRLGWQLDISYQLRAIAYMAKRVQNLELFLFCEDLEFVQNLDLGYPFVDMTTRDGAAHWDMMLMQSCKHGIITNSTYSWWAAYLIKNPEKIIIGPSHWIYGNENILCKDWVKIESQFETKS

SEQ ID NO: 14 - DNA, 858 bp, artificial
  Hybrid sequence of two organisms
  futC*/futL-Delta8 (1)..(858)
atggcctttaaagttgttcagatttgtggtggtctgggcaatcagatgtttcagtatgcatttgcaaaaagcctgcaaaagcatctgaatacaccggttctgctggatattaccagctttgattggagcaatcgtaaaatgcagctggaactgtttccgattgatctgccgtatgcaagcgcaaaagaaattgccattgcaaagatgcagcatctgccgaaactggttcgtgatgcactgaaatgtatgggttttgatcgtgtgagccaagaaatcgtgtttgaatatgaaccgaaactgctgaaaccgagccgtctgacctattttttcggttattttcaagatccgcgttacttcgatgcaattagtccgctgattaaacagacctttacactgcctccgcctccggaaaataacaaaaacaacaacaaaaaagaggaagagtactcacgtaaactgagccagattttagccgccaagaacagtgtgttcgtgcacattcgtcgcggcgactatatgcgtttaggctggcaattagacattagctatcaattacgcgctattgcttacatggccaagcgtgttcagaatctggagctgtttctgttctgcgaggatttagagtttgtgcagaatttagatttaggttatccgttcgtggacatgaccacacgcgatggtgccgcccactgggacatgatgctgatgcagagctgcaaacatggcatcatcaccaacagcacctatagttggtgggccgcctatttaatcaaaaatccggagaagatcatcatcggcccgagccattggatttacggcaacgagaatattctgtgtaaagattgggttaaaatttaa

SEQ ID NO: 15 - protein, 285 aa, artificial
  Hybrid sequence of two organisms
  FutC*/FutL-Delta8 (1)..(285)
MAFKVVQICGGLGNQMFQYAFAKSLQKHLNTPVLLDITSFDWSNRKMQLELFPIDLPYASAKEIAIAKMQHLPKLVRDALKCMGFDRVSQEIVFEYEPKLLKPSRLTYFFGYFQDPRYFDAISPLIKQTFTLPPPPENNKNNNKKEEEYSRKLSQILAAKNSVFVHIRRGDYMRLGWQLDISYQLRAIAYMAKRVQNLELFLFCEDLEFVQNLDLGYPFVDMTTRDGAAHWDMMLMQSCKHGIITNSTYSWWAAYLIKNPEKIIIGPSHWIYGNENILCKDWVKI

SEQ ID NO: 16 - DNA, 837 bp, artificial
  Hybrid sequence of two organisms
  futC*/futL-Delta15 (1)..(837)
atggcctttaaagttgttcagatttgtggtggtctgggcaatcagatgtttcagtatgcatttgcaaaaagcctgcaaaagcatctgaatacaccggttctgctggatattaccagctttgattggagcaatcgtaaaatgcagctggaactgtttccgattgatctgccgtatgcaagcgcaaaagaaattgccattgcaaagatgcagcatctgccgaaactggttcgtgatgcactgaaatgtatgggttttgatcgtgtgagccaagaaatcgtgtttgaatatgaaccgaaactgctgaaaccgagccgtctgacctattttttcggttattttcaagatccgcgttacttcgatgcaattagtccgctgattaaacagacctttacactgcctccgcctccggaaaataacaaaaacaacaacaaaaaagaggaagagtactcacgtaaactgagccagattttagccgccaagaacagtgtgttcgtgcacattcgtcgcggcgactatatgcgtttaggctggcaattagacattagctatcaattacgcgctattgcttacatggccaagcgtgttcagaatctggagctgtttctgttctgcgaggatttagagtttgtgcagaatttagatttaggttatccgttcgtggacatgaccacacgcgatggtgccgcccactgggacatgatgctgatgcagagctgcaaacatggcatcatcaccaacagcacctatagttggtgggccgcctatttaatcaaaaatccggagaagatcatcatcggcccgagccattggatttacggcaacgagaatattctgtaa

SEQ ID NO: 17 - protein, 278 aa, artificial
  Hybrid sequence of two organisms
  FutC*/FutL-Delta15 (1)..(278)
MAFKVVQICGGLGNQMFQYAFAKSLQKHLNTPVLLDITSFDWSNRKMQLELFPIDLPYASAKEIAIAKMQHLPKLVRDALKCMGFDRVSQEIVFEYEPKLLKPSRLTYFFGYFQDPRYFDAISPLIKQTFTLPPPPENNKNNNKKEEEYSRKLSQILAAKNSVFVHIRRGDYMRLGWQLDISYQLRAIAYMAKRVQNLELFLFCEDLEFVQNLDLGYPFVDMTTRDGAAHWDMMLMQSCKHGIITNSTYSWWAAYLIKNPEKIIIGPSHWIYGNENIL

SEQ ID NO: 18 - DNA, 624 bp, E.coli
  rcsA (1)..(624)
atgtcaacgattattatggatttatgtagttacacccgactaggtttaaccgggtatctgttgagtagaggggttaaaaaaagagaaatcaacgacattgaaaccgttgatgaccttgccatagcttgtgattcacagcgcccttcagtggtgtttattaatgaggactgtttcatccacgatgcttctaacagtcagcgtatcaagctcatcattaatcaacatcccaatacgttatttatcgtttttatggcaattgccaatgttcattttgatgaatatctattggtcagaaaaaatttattgatcagttctaaatcgattaaaccggaatctctcgacgatatccttggcgatattctgaaaaaagagacaacgataacctcgtttttaaatatgccgacgttatcattgagccgaaccgaatcgagtatgttgcgaatgtggatggcaggtcagggaaccattcaaatctctgaccaaatgaatatcaaagccaagaccgtttcatcgcataaaggtaatattaaacgtaagatcaaaacgcataataaacaggttatctaccatgtcgtccgactgacggataatgtgactaatggtatttttgtcaacatgcgctaa

SEQ ID NO: 19 - protein, 207 aa, E.coli
  RcsA (1)..(207)
MSTIIMDLCSYTRLGLTGYLLSRGVKKREINDIETVDDLAIACDSQRPSVVFINEDCFIHDASNSQRIKLIINQHPNTLFIVFMAIANVHFDEYLLVRKNLLISSKSIKPESLDDILGDILKKETTITSFLNMPTLSLSRTESSMLRMWMAGQGTIQISDQMNIKAKTVSSHKGNIKRKIKTHNKQVIYHVVRLTDNVTNGIFVNMR

SEQ ID NO: 20 - DNA, 46 bp, artificial
  Primer sequence
  futC/futC*fw (1)..(46)
ccccgaattctaaggaggaaattatatggcctttaaagttgttcag

SEQ ID NO: 21 - DNA, 38 bp, artificial
  Primer sequence
  futC/futC*rv (1)..(38)
cccctctagattattaggcgttatatttctggcttttc

SEQ ID NO: 22 - DNA, 47 bp, artificial
  Primer sequence
  futLfw (1)..(47)
ccccgaattctaaggaggaaattatatggattttaagatcgtgcaag

SEQ ID NO: 23 - DNA, 36 bp, artificial
  Primer sequence
  futLrv (1)..(36)
cccctctagattattaacttttggtctcaaactggc

SEQ ID NO: 24 - DNA, 50 bp, artificial
  Primer sequence
  rcsAfw (1)..(50)
ccccgctagcccctgcaaggaggtagaatgtcaacgattattatggattt

SEQ ID NO: 25 - DNA, 36 bp, artificial
  Primer sequence
  rcsArv (1)..(36)
cccctctagattattagcgcatgttgacaaaaatac

SEQ ID NO: 26 - DNA, 70 bp, artificial
  Primer sequence
  wcaJ-del-fw (1)..(70)
caatcgccgaccacttcgcgccgctgatggttttttcacgtaagctcataatgggaattagccatggtcc

SEQ ID NO: 27 - DNA, 70 bp, artificial
  Primer sequence
  wcaJ-del-rv (1)..(70)
ggatcttcccttaccccactgcgggtaaggggctaataacaggaacaacggtgtaggctggagctgcttc

SEQ ID NO: 28 - DNA, 17 bp, E.coli
  wcaJ-check-fw (1)..(17)
cgccgaccacttcgcgc

SEQ ID NO: 29 - DNA, 19 bp, E.coli
  wcaJ-check-rv (1)..(19)
atgtgccgctgatggaagc

SEQ ID NO: 30 - DNA, 75 bp, artificial
  Primer sequence
  lon-del-fw (1)..(75)
cacagtcgtgtcatctgattacctggcggaaattaaactaagagagagctctatgatgggaattagccatggtcc

SEQ ID NO: 31 - DNA, 70 bp, artificial; identical to SEQ ID NO: 27
  Primer sequence
  lon-del-rv (1)..(70)

SEQ ID NO: 32 - DNA, 21 bp, E.coli
  lon-check-fw (1)..(21)
cgtacatgttaatagatggcg

SEQ ID NO: 33 - DNA, 18 bp, E.coli
  lon-check-rv (1)..(18)
gccatctaacttagcgag

SEQ ID NO: 34 - DNA, 70 bp, artificial
  Primer sequence
  sulA-del-fw (1)..(70)
atgtacacttcaggctatgcacatcgttcttcgtcgttctcatccgcagcgtgtaggctggagctgcttc

SEQ ID NO: 35 - DNA, 71 bp, artificial
  Primer sequence
  sulA-del-rv (1)..(71)
ttaatgatacaaattagagtgaatttttagcccggaaagttgtctcgtggtccgtcgacctgcagttcgaa

SEQ ID NO: 36 - DNA, 18 bp, E.coli
  sulA-check-fw (1)..(18)
gccagtgccactgcaatc

SEQ ID NO: 37 - DNA, 18 bp, E.coli
  sulA-check-rv (1)..(18)
cgcaaacaccgtgtcgtc

SEQ ID NO: 38 - DNA, 47 bp, artificial
  Primer sequence
  lac-1-fw (1)..(47)
cgcttgggcaagccgcaggatcctttgctgatgcctgctatggctcg

SEQ ID NO: 39 - DNA, 34 bp, artificial
  Primer sequence
  lac-2-rv (1)..(34)
gaagtcgcttaaattataaaaattgcctgatacg

SEQ ID NO: 40 - DNA, 36 bp, artificial
  Primer sequence
  lac-3-fw (1)..(36)
ggcaatttttataatttaagcgacttcattcacctg

SEQ ID NO: 41 - DNA, 36 bp, artificial
  Primer sequence
  lac-4-rv (1)..(36)
caggaaacagctatgtactatttaaaaaacacaaac

SEQ ID NO: 42 - DNA, 40 bp, artificial
  Primer sequence
  lac-5-fw (1)..(40)
gttttttaaatagtacatagctgtttcctgtgtgaaattg

SEQ ID NO: 43 - DNA, 51 bp, artificial
  Primer sequence
  lac-6-rv (1)..(51)
cgggaaggcgactggagtgccatggatccggttttcaacaaaccatgcaaa

SEQ ID NO: 44 - DNA, 20 bp, artificial
  Primer sequence
  lac-7-fw (1)..(20)
cgcaggatcctttgctgatg

SEQ ID NO: 45 - DNA, 20 bp, artificial
  Primer sequence
  lac-8-rv (1)..(20)
ccatggatccggttttcaac

SEQ ID NO: 46 - DNA, 20 bp, artificial
  Primer sequence
  pMAK-fw (1)..(20)
tcactatggcgtgctgctag

SEQ ID NO: 47 - DNA, 18 bp, artificial
  Primer sequence
  lac-9-rv (1)..(18)
tcgcattgggtcaccagc

SEQ ID NO: 48 - DNA, 18 bp, artificial
  Primer sequence
  lac-10-fw (1)..(18)
gccagcctgattgcctgg

SEQ ID NO: 49 - DNA, 20 bp, artificial
  Primer sequence
  pMAK-rv (1)..(20)
agatggcgcccaacagtccc

SEQ ID NO: 50 - DNA, 19 bp, E.coli
  lac-11-fw (1)..(19)
cggcgcaaactgttaatgc

SEQ ID NO: 51 - DNA, 19 bp, E.coli
  lac-12-rv (1)..(19)
gcaaatgctgaatgagggc

SEQ ID NO: 52 - DNA, 32 bp, artificial
  Primer sequence
  futL-Scafw (1)..(32)
gcacgcagagcagtactcacgtaaactgagcc

SEQ ID NO: 53 - DNA, 32 bp, artificial
  Primer sequence
  futL-Scarv (1)..(32)
ggctcagtttacgtgagtactgctctgcgtgc

SEQ ID NO: 54 - DNA, 26 bp, artificial
  Primer sequence
  C-futC*fw (1)..(26)
ccccgaattctaaggaggaaattata

SEQ ID NO: 55 - DNA, 32 bp, artificial
  Primer sequence
  C-futC*rv (1)..(32)
cgtgagtactcttcctcttttttgttgttgtt

SEQ ID NO: 56 - DNA, 26 bp, artificial
  Primer sequence
  N-futC*fw (1)..(26)
ccccgaattctaaggaggaaattata

SEQ ID NO: 57 - DNA, 32 bp, artificial; identical to SEQ ID NO: 55
  Primer sequence
  N-futC*rv (1)..(32)

SEQ ID NO: 58 - DNA, 25 bp, artificial
  Primer sequence
  futC*-short-fw (1)..(25)
cacaggaaacagaattctaaggagg

SEQ ID NO: 59 - DNA, 34 bp, artificial
  Primer sequence
  futC*-short8-rv (1)..(34)
aattttaacccaatctttacacagaatattctcg

SEQ ID NO: 60 - DNA, 48 bp, artificial
  Primer sequence
  rcsA-2-fw (1)..(48)
ctgtgtaaagattgggttaaaatttaataatctagcccctgcaaggag

SEQ ID NO: 61 - DNA, 26 bp, artificial
  Primer sequence
  rcsA-2-rv (1)..(26)
ccaagcttctagattattagcgcatg

SEQ ID NO: 62 - DNA, 26 bp, artificial
  Primer sequence
  futC*-short15-rv (1)..(26)
cagaatattctcgttgccgtaaatcc

SEQ ID NO: 63 - DNA, 50 bp, artificial
  Primer sequence
  rcsA-3-fw (1)..(50)
ggatttacggcaacgagaatattctgtaataatctagcccctgcaaggag